(19) United States
(12) Patent Application Publication
(10) Pub. No.: US 2018/0133248 A1
(43) Pub. Date: May 17, 2018

(54) THERAPEUTIC OZONE AGENT AND TREATMENT

(71) Applicant: James A. Caplan, Rosemont, PA (US)

(72) Inventor: James A. Caplan, Rosemont, PA (US)

(21) Appl. No.: 15/866,749

(22) Filed: Jan. 10, 2018

Related U.S. Application Data
(63) Continuation-in-part of application No. 14/594,481, filed on Jan. 12, 2015, now abandoned.
Application No. 14/594,481 is a continuation-in-part of application No. 14/578,733, filed on Dec. 22, 2014, now abandoned.

(51) Int. Cl. A61K 33/00; A61P 39/00

(52) U.S. Cl. CPC A61K 33/00; A61P 39/00

(57) ABSTRACT

The use of a medical therapeutic ozone agent in vivo to treat a variety of disorders including, but not limited to hypoxic conditions, autoimmune disease, fibrotic disease, inflammatory disease, neurodegenerative disease, infectious disease, lung disease, heart and vascular disease, metabolic disease, and cancer, wherein the therapeutic ozone agent is administered in a therapeutically effective amount, which can convert carbon monoxide to carbon dioxide, thereby treating or preventing the disease in a patient.

This application is a continuation in part of U.S. patent application Ser. No. 14/594,481, filed Jan. 12, 2015, which is a continuation in part of U.S. patent application Ser. No. 14/578,733 filed on Dec. 22, 2014, all of which are incorporated by reference herein in their entirety.

### BACKGROUND OF THE INVENTION

### 1. Field of Invention

This invention relates to the use of a medical therapeutic ozone agent in vivo to treat a variety of disorders including, but not limited to, hypoxic conditions, anemia, autoimmune disease, fibrotic disease, inflammatory disease, neurodegenerative disease, infectious disease, lung disease, heart and vascular disease, metabolic disease, and cancer, wherein the therapeutic ozone agent is administered in a therapeutically effective amount, which can convert carbon monoxide to carbon dioxide, thereby aiding recovery by allowing hemoglobin to carry more oxygen, treating or preventing the disease in a patient.

### 2. Description of Related Art

Numerous reports have been published on the safety and clinical results obtained by the application of the therapeutic ozone agent in diseases related to insufficient oxygen supply to tissues and various organs, and/or the disruption of its utilization in the cells. Ozone virucidal effect has been reported at dose levels at which no undesirable side effects take place, offering promise as a means to inactivate human retroviruses in human body fluids and blood products preparation.

Among the medical properties of ozone documented are the ability to increase the rate and capacity of oxygen absorption in erythrocytes and the activation of glycolisis in the cells via the pentose pathway. This enhances the production of 2,3 DPG, which is known to act as a coadjuvant of oxygen release from oxyhemoglobin at tissue level. (Wells K H., Latino J., Gavalchin J. Inactivation of human immunodeficiency virus type I by ozone in vitro. Blood, vol. 78:7, p. 1982, 1991; Balkanyi A. The interaction between ozone therapy and oxygen radicals in their importance in practice. Proceedings of the Ninth Ozone World Congress. New York. Jun. 3-9, 1989). Both effects lead to significant improvement in oxygen supply to the body, demonstrated in vivo by the measurement of pO₂ increase in arterial blood as well as the reduction in venous. (Baltin H. Oxygen partial pressure measurements in the arterial and venous blood before, during and after ozone treatment. OzoNews 2 (2):41, 1983).

In addition, the rheological properties of the blood improve, especially in regard to erythrocytes aggregation (preventing rouleaux formation and clumping) and membrane permeability and flexibility, because of the effect of ozone/oxygen on it. As a consequence of these effects, reduction of blood viscosity and enhancement of blood flow are achieved. (Rokitansky O. Clinical considerations and Biochemistry of ozone therapy. Hospitalis, 52,643, 1982; Gomez M., Menendez S. About ozone metabolic effects in biological systems. (Spa.). Conference presented in the International Congress Geriatrics/92, Palace of Conventions, Ciudad Habana, Mayo 1992; Mattasi, R. Ozonoterapia. Organizzazione Editoriale Medico Farmaceutica, Milano 1985, p 64).

Numerous preclinical experiments have been performed in vitro and in vivo to test possible ozone/oxygen toxicity related to the therapeutic methods and ways of administration according to which the therapeutic ozone agent is currently applied. Controlled in vitro testing on the degree of hemolysis and “Heinz Body Formation” induced by the administration of ozone to blood at adequate dosage was performed, (Hernandez F., Menendez S., Gomez M. Blood ozone treatment and the hemolysis level. X Ozone World Congress. Monaco, 1991) not finding any significant effect, neither on the hemolysis level nor in the resistance of erythrocytes to further oxidative stress. These results are consistent with the fact that ozone stimulates several enzymatic redox systems responsible for cells protection against oxidation. (Gomez M., Menendez S. About ozone metabolic effects in biological systems. (Spa.). Conference presented in the International Congress Geriatrics/92, Palace of Conventions, Ciudad Habana, Mayo 1992) Doses up to more than ten times the maximum therapeutic levels were tested for toxicity. These studies comprised cytotoxicity, organs function, hematological parameters, histological studies by electron microscopy, teratogenicity and cytogenetic testing. All the results demonstrated the non-toxic nature of the therapeutic ozone agent within the range of therapeutic dose levels when administered intravascularly, intramuscularly and intrarectally.

Carbon monoxide can enter the body exogenously by inhalation (for example, by inhalation of smoke) or can be produced endogenously by the process of hemolysis (the destruction of red blood cells). Carbon monoxide bonds to the oxygen receptors on the hemoglobin molecule with a binding force of some 210 to 250 times the binding constant of oxygen, which can make it impossible for those carbon monoxide bound receptors to carry oxygen.

Sickle cell anemia, a genetic disease which involves the sickle shape of erythrocytes, can be exacerbated by the presence of CO. In sickle cell disease, a mutated hemoglobin, HbS, which has a substitution of glutamic acid by valine in the amino acid chain, causes a change in the shape of red blood cells when blood oxygen tension is low, The crystallization or intracellular polymerization of the molecules of HbS occurs when those cells are deprived of oxygen up to a partial pressure of oxygen (pO₂) below the threshold level at which the cells sickle. In these conditions, the erythrocytes lose their normal elasticity and shape, also losing their capacity to take and deliver oxygen and increasing the viscosity of the blood. This leads to a reduction in the availability of oxygen to the cells, producing painful crisis, infarction, abdominal and/or muscular pain, ulcers, etc.

This process is reversible in the early stages, for when the HbS molecule is reoxygenated the cell distortion disappears and resumes its normal shape. The longer the period of time necessary for the reoxygenation of the molecules of HbS, the greater number of red cells die. Therefore, the increase in the partial pressure of oxygen and the promptness with which it is normalized are determining factors for the symptoms to diminish and disappear. Attempts have been made to establish effective treatment for this disease, but their value and absence of side effects has not been confirmed in medical practice. (Gomez, M., Espinosa, E., and Caplan, J. A., 1995, Application of medicinal ozone/oxygen in patients with sickle cell anemia, Townsend Lett. Doctors January: 48-52).

In addition, the free carbon monoxide circulating in the plasma is in search of a binding position or at least one of the four oxygen receptors on hemoglobin. The circulating and as yet unbonded CO is a constant threat to the body because it always has the potential to find a bonding place on a hemoglobin oxygen receptor, which makes it impossible for that receptor to again carry oxygen. The greater percentage of oxygen receptors bonded by CO the greater the degradation of the body due to oxygen deprivation of the tissues, with the end point thing death at high levels of CO poisoning.

The inventor has found that a therapeutic ozone agent can be administered in a therapeutically effective amount, which can convert carbon monoxide to carbon dioxide, thereby treating or preventing diseases in a patient.

All references cited herein are incorporated herein by reference in their entireties.

### BRIEF SUMMARY OF THE INVENTION

The invention provides a method for treating or preventing a disease in a patient, wherein the disease is selected from the group consisting of autoimmune disease, fibrotic disease, inflammatory disease, neurodegenerative disease, infectious disease, lung disease, heart and vascular disease, metabolic disease, cancer, and combinations thereof, wherein said method comprises: selecting a patient in need of treating or preventing said disease; administering to the patient at least one therapeutic ozone agent in a therapeutically effective amount, wherein the at least one therapeutic ozone agent is administered in a concentration to convert carbon monoxide bound to hemoglobin to carbon dioxide, thereby treating or preventing said disease in said patient. The invention provides a method wherein the at least one therapeutic ozone agent is administered by a method selected from the group consisting of rectal insufflation, extracorporeal treatment, re-administration of the patient's blood, intramuscular administration, cardiopulmonary bypass, and combinations thereof. The invention provides a method wherein the at least one therapeutic ozone agent is administered in a form selected from the group consisting of a gas, a liquid formulation, a combination with O2, and combinations thereof.

The invention provides a method for treating or preventing a disease in a patient, wherein the disease is selected from the group consisting of autoimmune disease, fibrotic disease, inflammatory disease, neurodegenerative disease, infectious disease, lung disease, heart and vascular disease, metabolic disease, cancer, and combinations thereof wherein said method comprises: selecting a patient in need of treating or preventing said disease; administering to the patient at least one therapeutic ozone agent in a therapeutically effective amount, wherein the at least one therapeutic ozone agent is administered in a concentration to convert carbon monoxide dissolved in plasma to carbon dioxide, thereby treating or preventing said disease in said patient. The invention provides a method wherein the at least one therapeutic ozone agent is administered by a method selected from the group consisting of rectal insufflation, extracorporeal treatment and re-administration of the patient's blood, intramuscular administration, cardiopulmonary bypass, and combinations thereof. The invention provides a method wherein the at least one therapeutic ozone agent is administered in a form selected from the group consisting of a gas, a liquid formulation, a combination with O₂, and combinations thereof.

The invention provides a method for treating or preventing carbon monoxide poisoning in a patient wherein said method comprises: selecting a patient in need of treating or preventing carbon monoxide poisoning; administering to the patient in need of treating or preventing carbon monoxide poisoning at least one therapeutic ozone agent in a therapeutically effective amount. The invention provides a method wherein the carbon monoxide poisoning is selected from the group consisting of acute, chronic, and combinations thereof. The invention provides a method wherein the a therapeutic ozone agent is administered by a method selected from the group consisting of rectal insufflation, extracorporeal treatment and re-administration of the patient's blood, intramuscular administration, cardiopulmonary bypass, and combinations thereof. The invention provides a method wherein the at least one therapeutic ozone agent is administered in a form selected from the group consisting of a gas, a liquid formulation, a combination with O₂, and combinations thereof.

The invention provides a method for treating or preventing of one or more manifestations or complications that can lead to intravascular sickling and vaso-occlusion in sickle cell disease in a patient, wherein said method comprises: selecting a patient in need of treating or preventing one or more manifestations or complications that can lead to intravascular sickling and vaso-occlusion in sickle cell disease; administering to the patient a therapeutic ozone agent in a therapeutically effective amount; and administering to the patient at least one additional active agent. The invention provides a method where the manifestation or complication is one or more selected from the group consisting of ischemia reperfusion injury, acute chest syndrome, especially acute chest injury, hyposplenism, asplenism, cardiovascular damages, infection, or another manifestation of SCD. The invention provides a method wherein the at least one therapeutic ozone agent is administered in a form selected from the group consisting of a gas, a liquid formulation, a combination with O₂, and combinations thereof. The invention provides a method wherein the at least one additional active agent is selected from the group consisting of antibiotics, analgesics, narcotics, hydroxyurea, anticoagulants, supplemental oxygen, stem cell transplant, gene therapy, nitric oxide, statins, and combinations thereof. The invention provides a method wherein the patient is an adult. The invention provides a method wherein the patient is a pediatric patient. The invention provides a method wherein the ozone is administered by a method selected from the group consisting of rectal insufflation, extracorporeal treatment and re-administration of the patient's blood, intramuscular administration, cardiopulmonary bypass, and combinations thereof.

The invention provides a method for treating or preventing anemia, which can be considered the premature death of red blood cells, in a patient wherein said method comprises: selecting a patient in need of treating or preventing anemia; administering to the patient in need of treating or preventing anemia at least one a therapeutic ozone agent in a therapeutically effective amount. The invention provides a method wherein the anemia is selected from the group consisting of iron deficiency anemia, Thalassemia, Aplastic anemia, Hemolytic anemia, Sickle cell anemia, Pernicious anemia, Fanconi anemia, and combinations thereof. The invention provides a method wherein the a therapeutic ozone agent is administered by a method selected from the group consisting of rectal insufflation, extracorporeal treatment and re-administration of the patient's blood, intramuscular administration, cardiopulmonary bypass, and combinations thereof. The invention provides a method wherein the at least one therapeutic ozone agent is administered in a form selected from the group consisting of a gas, a liquid formulation, a combination with O₂, and combinations thereof.

The invention provides a method for improving self-renewal, growth, mobilization, proliferation and/or differentiation of a therapeutic cell composition in a patient, wherein said method comprises: selecting a patient who is going to receive a therapeutic cell composition; administering to the patient a therapeutic cell composition; administering to the patient at least one therapeutic ozone agent in a therapeutically effective amount, wherein the at least one therapeutic ozone agent is administered in a concentration to convert carbon monoxide dissolved in plasma to carbon dioxide, thereby improving the self-renewal, growth, mobilization, proliferation and/or differentiation of the therapeutic cell composition in said patient. The invention provides a method wherein the at least one therapeutic ozone agent is administered by a method selected from the group consisting of rectal insufflation, extracorporeal treatment and re-administration of the patient's blood, intramuscular administration, cardiopulmonary bypass, and combinations thereof. The invention provides a method wherein the at least one therapeutic ozone agent is administered in a form selected from the group consisting of a gas, a liquid formulation, a combination with O2, and combinations thereof. The invention provides a method wherein the therapeutic cell composition comprises cells selected from the group consisting of stem cells, totipotent cells, progenitor cell, embryonic stem cells, pluripotent stem cells, adult stem cells, induced pluripotent stem cells, genetically modified cells, and combinations thereof.

The invention provides a method for improving the self-renewal, growth, mobilization, proliferation and/or differentiation of a therapeutic cell composition in a patient, wherein said method comprises: selecting a patient who has received a therapeutic cell composition; administering to the patient at least one therapeutic ozone agent in a therapeutically effective amount, wherein the at least one therapeutic ozone agent is administered in a concentration to convert carbon monoxide dissolved in plasma to carbon dioxide, thereby improving the self-renewal, growth, mobilization, proliferation and/or differentiation of the therapeutic cell composition in said patient. The invention provides a method wherein the at least one therapeutic ozone agent is administered by a method selected from the group consisting of rectal insufflation, extracorporeal treatment and re-administration of the patient's blood, intramuscular administration, cardiopulmonary bypass, and combinations thereof. The invention provides a method wherein the at least one therapeutic ozone agent is administered in a form selected from the group consisting of a gas, a liquid formulation, a combination with O₂, and combinations thereof. The invention provides a method wherein the therapeutic cell composition comprises cells selected from the group consisting of stem cells, totipotent cells, progenitor cell, embryonic stem cells, pluripotent stem cells, adult stem cells, induced pluripotent stem cells, genetically modified cells, and combinations thereof.

The invention provides a method for improving the self-renewal, growth, mobilization, proliferation and/or differentiation of a therapeutic cell composition in a patient, wherein said method comprises: selecting a patient who is going to receive a therapeutic cell composition; administering to the patient a therapeutic cell composition; administering to the patient at least one therapeutic ozone agent in a therapeutically effective amount, wherein the at least one therapeutic ozone agent is administered in a concentration to convert carbon monoxide bound to hemoglobin to carbon dioxide, thereby improving the self-renewal, growth, mobilization, proliferation and/or differentiation of the therapeutic cell composition in said patient. The invention provides a method wherein the at least one therapeutic ozone agent is administered by a method selected from the group consisting of rectal insufflation, extracorporeal treatment and re-administration of the patient's blood, intramuscular administration, cardiopulmonary bypass, and combinations thereof. The invention provides a method wherein the at least one therapeutic ozone agent is administered in a form selected from the group consisting of a gas, a liquid formulation, a combination with O2, and combinations thereof. The invention provides a method wherein the therapeutic cell composition comprises cells selected from the group consisting of stem cells, totipotent cells, progenitor cell, embryonic stem cells, pluripotent stem cells, adult stem cells, induced pluripotent stem cells, genetically modified cells, and combinations thereof.

The invention provides a method for improving the self-renewal, growth, mobilization, proliferation and/or differentiation of a therapeutic cell composition in a patient, wherein said method comprises: selecting a patient who has received a therapeutic cell composition; administering to the patient at least one therapeutic ozone agent in a therapeutically effective amount, wherein the at least one therapeutic ozone agent is administered in a concentration to convert carbon monoxide bound to hemoglobin to carbon dioxide, thereby improving the self-renewal, growth, mobilization, proliferation and/or differentiation of the therapeutic cell composition in said patient. The invention provides a method wherein the at least one therapeutic ozone agent is administered by a method selected from the group consisting of rectal insufflation, extracorporeal treatment and re-administration of the patient's blood, intramuscular administration, cardiopulmonary bypass, and combinations thereof. The invention provides a method wherein the at least one therapeutic ozone agent is administered in a form selected from the group consisting of a gas, a liquid formulation, a combination with O₂, and combinations thereof. The invention provides a method wherein the therapeutic cell composition comprises cells selected from the group consisting of stem cells, totipotent cells, progenitor cell, embryonic stem cells, pluripotent stem cells, adult stem cells, induced pluripotent stem cells, genetically modified cells, and combinations thereof.

The invention provides a method for prevention and/or treatment of Alzheimer's disease in a patient, wherein said method comprises: selecting a patient in need of prevention and/or treatment of Alzheimer's Disease; administering to the patient at least one therapeutic ozone agent in a therapeutically effective amount, wherein the at least one therapeutic ozone agent is administered in a concentration to convert carbon monoxide dissolved in plasma to carbon dioxide, thereby preventing and/or treating Alzheimer's Disease in said patient. The invention provides a method wherein the at least one therapeutic ozone agent is administered by a method selected from the group consisting of rectal insufflation, extracorporeal treatment and re-administration of the patient's blood, intramuscular administration, cardiopulmonary bypass, and combinations thereof. The invention provides a method wherein the at least one therapeutic ozone agent is administered in a form selected from the group consisting of a gas, a liquid formulation, a combination with O₂, and combinations thereof.

The invention provides a method for prevention and/or treatment of Alzheimer's disease in a patient, wherein said method comprises: selecting a patient in need of prevention and/or treatment of Alzheimer's Disease; administering to the patient at least one therapeutic ozone agent in a therapeutically effective amount, wherein the at least one therapeutic ozone agent is administered in a concentration to convert carbon monoxide bound to hemoglobin to carbon dioxide, thereby preventing and/or treating Alzheimer's Disease in said patient. The invention provides a method wherein the at least one therapeutic ozone agent is administered by a method selected from the group consisting of rectal insufflation, extracorporeal treatment and re-administration of the patient's blood, intramuscular administration, cardiopulmonary bypass, and combinations thereof. The invention provides a method wherein the at least one therapeutic ozone agent is administered in a form selected from the group consisting of a gas, a liquid formulation, a combination with O₂, and combinations thereof.

The invention provides a method for treating carbon monoxide poisoning in a patient wherein said method comprises: selecting a patient in need of treatment of carbon monoxide poisoning; administering to the patient in need of treatment of carbon monoxide poisoning a therapeutic ozone agent in a therapeutically effective amount, wherein the therapeutically effective amount of the therapeutic ozone agent is administered in a concentration to convert carbon monoxide dissolved in the patient's plasma to carbon dioxide, and also to convert carbon monoxide bonded to the patient's hemoglobin receptors to carbon dioxide, wherein the ozone is administered over a period of time sufficient to diminish CO levels in the plasma, further wherein the ozone administered causes a rise in PO₂ from between the range of 2% to 30% thereby treating said carbon monoxide poisoning in said patient.

The invention provides a method for treating carbon monoxide poisoning in a patient wherein the therapeutic ozone agent is ozone alone.

The invention provides a method for treating carbon monoxide poisoning in a patient, wherein the carbon monoxide poisoning is selected from the group consisting of acute, chronic, and combinations thereof.

The invention provides a method for treating carbon monoxide poisoning in a patient, wherein the a therapeutic ozone agent is administered by a method selected from the group consisting of rectal insufflation, extracorporeal treatment and re-administration of the patient's blood, intramuscular administration, cardiopulmonary bypass, and combinations thereof.

The invention provides a method for treating carbon monoxide poisoning in a patient, wherein the at least one therapeutic ozone agent is administered in a form selected from the group consisting of a gas, a liquid formulation, a combination with O₂, and combinations thereof.

The invention provides a method for treating carbon monoxide poisoning in a patient wherein the dose therapeutically effective amount of therapeutic ozone administered is varied according to individual's sensitivity and the type of disease state being treated.

### DETAILED DESCRIPTION OF THE INVENTION

Herewith is presented a method of treating carbon monoxide bonded hemoglobin and carbon monoxide dissolved in blood plasma with a therapeutic ozone agent, converted, for example, from medical grade oxygen. The therapeutic ozone at non-toxic levels is administered periodically such that, firstly, the carbon monoxide in plasma is proportionally converted to carbon dioxide and, secondly some amount of carbon monoxide bonded to hemoglobin receptors is converted to carbon dioxide. That is, assuming carbon monoxide bonded hemoglobin is not first destroyed by hemolysis. The resultant CO₂ passing out of the body during the respiratory cycle.

Thus, over time the plasma is proportionally freed of the carbon dioxide and the threat of carbon monoxide bonding to hemoglobin is reduced in proportion to the amount of carbon monoxide that is converted to CO₂ and some amount of carbon monoxide bonded hemoglobin is freed of the carbon monoxide. The sum total effect of the administration of ozone is that a greater amount of hemoglobin can carry and deliver oxygen to the tissues posttreatment then pretreatment.

As used herein, “comprising,” “including,” “containing,” “characterized by,” and grammatical equivalents thereof are inclusive or open-ended terms that do not exclude additional, unrecited elements or method steps, but also include the more restrictive terms “consisting of” and “consisting essentially of.”

As used herein and in the appended claims, the singular forms, for example, “a”, “an”, and “the,” include the plural, unless the context clearly dictates otherwise. For example, reference to “a gas-fluid contacting device” includes a plurality of such devices, and reference, for example, to a “protein” is a reference to a plurality of similar proteins, and equivalents thereof.

As used herein, the terms “subject” and “patient” are used interchangeably. As used herein, the term “patient” refers to an animal, preferably a mammal such as a non-primate (e.g., cows, pigs, horses, cats, dogs, rats etc.) and a primate (e.g., monkey and human), and most preferably a human. In some embodiments, the subject is a non-human animal such as a farm animal (e.g., a horse, pig, or cow) or a pet (e.g., a dog or cat). In a specific embodiment, the subject is an elderly human. In another embodiment, the subject is a human adult. In another embodiment, the subject is a human child. In yet another embodiment, the subject is a human infant.

“Absorption of ozone by a biological fluid” is defined as the phenomenon wherein ozone reacts with the fluid being treated by a variety of mechanisms, including oxidation. Regardless of the mechanism involved, the reaction occurs instantaneously.

A “biological fluid” is defined as a composition originating from a biological organism of any type. Examples of biological fluids include blood, blood products and other fluids, such as saliva, urine, feces, semen, milk, tissue, tissue samples, homogenized tissue samples, gelatin, and any other substance having its origin in a biological organism. Biological fluids may also include synthetic materials incorporating a substance having its origin in a biological organism, such as a vaccine preparation containing alum and a virus (the virus being the substance having its origin in a biological organism), cell culture media, cell cultures, viral cultures, and other cultures derived from a biological organism.

A “blood product” is defined as including blood fractionates and therapeutic protein compositions containing proteins derived from blood. Fluids containing biologically active proteins other than those derived from blood may also be treated by the method.

“In vivo” use of a material or compound is defined as the introduction of a material or compound into a living human, mammal or vertebrate.

“In vitro” use of a material or compound is defined as the use of the material or compound outside a living human, mammal or vertebrate, where neither the material nor compound is intended for reintroduction into a living human, mammal or vertebrate. An example of an in vitro use would be the analysis of a component of a blood sample using laboratory equipment.

“Ex vivo” use of a process is defined as using a process for treatment of a biological material such as a blood product outside of a living human, mammal or vertebrate. For example, removing blood from a human and subjecting that blood to a method to treat acute ischemic brain stroke is defined as an ex vivo use of that method if the blood is intended for reintroduction into that human or another human. Reintroduction of the human blood into that human or another human would be an in vivo use of the blood, as opposed to an ex vivo use of the method.

“Extracorporeal” is defined as a state wherein blood or blood fractionate is treated outside (ex vivo) of the body, for example, in the delivery of a measured amount of ozone to a sample of patient's blood.

“Synthetic media” is defined as an aqueous synthetic blood or blood product storage media.

A “pharmaceutically-acceptable carrier” or “pharmaceutically-acceptable vehicle” is defined as any liquid including water, saline, a gel, salve, solvent, diluent, fluid ointment base, liposome, micelle and giant micelle, which is suitable for use in contact with a living animal or human tissue without causing adverse physiological responses, and which does not interact with the other components of the composition in a deleterious manner.

“Biologically active” is defined as capable of effecting a change in the living organism or component thereof.

This aim is achieved, according to the present invention, by the use of a therapeutic ozone agent for the treatment or prevention of a disease or condition selected from the group consisting of, for example, anemia, autoimmune disease, fibrotic disease, inflammatory disease, neurodegenerative disease, infectious disease, lung disease, heart and vascular disease, metabolic disease, cancer, carbon monoxide poisoning, and anemia.

Surprisingly and in contrast to the state of the art, it could be shown that a therapeutic ozone agent is suitable for the treatment or prevention of autoimmune disease, fibrotic disease, inflammatory disease, neurodegenerative disease, infectious disease, lung disease, heart and vascular disease, metabolic disease, cancer, carbon monoxide poisoning, and anemia.

In one preferred embodiment, the therapeutic ozone agent is used for the treatment or prevention of autoimmune disease, fibrotic disease, inflammatory disease, neurodegenerative disease, infectious disease, lung disease, heart and vascular disease, metabolic disease, cancer, carbon monoxide poisoning, and anemia.

The term “solution” refers to a liquid mixture and the term “aqueous solution” refers to a solution that contains some water and may also contain one or more other liquid substances with water to form a multi-component solution.

### Hemoglobin

Blood consists of 55% plasma (water, dissolved salts and proteins), 45% red cells and a smattering of white cells. Virtually all the oxygen carried by the blood is bound to hemoglobin in the red cells (erythrocytes). There are about 280 million red-colored hemoglobin molecules within each of the 1013 erythrocytes circulating in the blood. Each molecule of hemoglobin can carry as many as four molecules of oxygen and hemoglobin can transport over 70 times more oxygen than can be simply dissolved in the blood. Bright red hemoglobin that is 100% saturated with oxygen is called oxyhemoglobin (HbO). Hemoglobin without oxygen is called deoxyhemoglobin (or simply Hb) and is much darker and bluish.

Hemoglobin is a tetrameric protein which delivers oxygen via an allosteric mechanism. Oxygen binds to the four hemes of the hemoglobin molecule. Each heme contains porphyrin and iron in the ferrous state. The ferrous iron-oxygen bond is readily reversible. Binding of the first oxygen to a heme releases much greater energy than binding of the second oxygen molecule, binding of the third oxygen releases even less energy, and binding of the fourth oxygen releases the least energy.

In blood, hemoglobin is in equilibrium between two allosteric structures. In the “T” (for tense) state, hemoglobin is deoxygenated. In the “R” (for relaxed) state, hemoglobin is oxygenated. An oxygen equilibrium curve can be scanned to observe the affinity and degree of cooperativity (allosteric action) of hemoglobin. In an oxygen equilibrium curve, the Y-axis plots the percent of hemoglobin oxygenation and the X-axis plots the partial pressure of oxygen in millimeters of mercury (mm Hg). If a horizontal line is drawn from the 50% oxygen saturation point to the scanned curve and a vertical line is drawn from the intersection point of the horizontal line with the curve to the partial pressure X-axis, a value commonly known as the P 50 is determined (i.e., this is the pressure in mm Hg when the scanned hemoglobin sample is 50% saturated with oxygen). Under physiological conditions (i.e., 37 C., pH=7.4, and partial carbon dioxide pressure of 40 mm Hg), the P₅₀ value for normal adult hemoglobin (HbA) is around 26.5 mm Hg. If a lower than normal P₅₀ value is obtained for the hemoglobin being tested, the scanned curve is considered to be “left-shifted”and the presence of high oxygen-affinity hemoglobin is indicated. Conversely, if a higher than normal P 50 value is obtained for the hemoglobin being tested, the scanned curve is considered to be “right-shifted”, indicating the presence of low oxygen-affinity hemoglobin.

At sea level pressure (760 mm Hg) the 21% oxygen in air has a partial pressure (pO₂) of 160 mm of mercury. If air is bubbled through blood, the hemoglobin becomes about 100% saturated with oxygen. The saturation of hemoglobin is not linear with the partial pressure of oxygen but “s” shaped, rising very slowing at low pO₂ and then more rapidly as the oxygen tension approaches 20 mm where the slope becomes steepest. By 60 mm the saturation is already up to 90 “SAT” (percentage of full saturation). This relationship, called the “oxygen dissociation curve” is not fixed, but highly affected by other constituents such as carbon dioxide, acidity (pH) and temperature. At 30 mm oxygen tension, a 10 mm increase in CO₂ tension will lower oxygen saturation by about 4 SAT units as will a 0.1 pH decrease or 2° C. temperature increase.

Hemoglobin takes on a maximal load of oxygen over a wide range of atmospheric oxygen partial pressures and carries it to the tissues of the body where oxygen is being consumed and the O₂ tension is low. As tissue pO₂ falls, the hemoglobin is forced to give up some of its oxygen making it available to the tissue. The greater the local metabolism, the lower the tissue oxygen tension. In rapidly metabolizing tissue, the O₂ tension falls to the steep part of the dissociation curve and much more oxygen is released by the hemoglobin.

The oxygen-depleted venous blood flows back to the lungs where the hemoglobin again becomes saturated with oxygen from the alveoli of the lungs.

In order to transport oxygen to cells, there needs to be at least a 20 mm (Hg) gradient between the oxygen-using part of the cell, i.e. mitochondria and the hemoglobin in the capillaries. When pO₂ falls to this value little oxygen can be transferred even though the hemoglobin is still 25% saturated (Landis E M, Pappenheimer J R, “Exchange of substances through the capillary walls”, Chapter 29, Handbook of Physiology, Circulation, Vol. II (Hamilton W F, editor), 1963, American Physiological Society, Washington, D.C.) Anaerobic degradation of glucose to lactate (glycolysis) is an emergency way of producing chemical energy (ATP) when the oxygen supply is cut off. In fact, red cells are always anaerobic. They do not use oxygen but depend entirely on glycolysis for their chemical energy.

Assume deoxyHb-T is at zero pO₂ and gradually increase the oxygen tension. At low pressures, very little oxygen will be absorbed because of the very low oxygen affinity of tight hemoglobin. This is the “toe” of the dissociation curve. As pO₂ increases, some of the Hb-T molecules will begin to add a molecule of oxygen. As pressure continues to increase, more hemoglobin molecules will bind an oxygen molecule. At some point, the pressure will be high enough for some hemoglobin molecules to bind a second oxygen. As the number of bound oxygen molecules increase so does the tendency for hemoglobin to snap from Hb-T to Hb-R. Once the change takes place to the relaxed form of the hemoglobin molecule, its affinity for oxygen is so great that it quickly fills all four of its heme sites. One of the factors driving the conversion may be the tension of the bound oxygen molecule pulling on the iron atom in the heme group. Some hemoglobin molecules convert on adding the second oxygen and others wait until the third. Hb-T cannot exist with four oxygen molecules. Other coexisting conditions affect the conversion point. As pO₂ increases, a crescendo of clicking Hb-T to Hb-R conversions occurs giving rise to the steep part of the oxygen dissociation curve.

The reverse process occurs. Arterial hemoglobin is almost entirely oxyhemoglobin (Hb-R). As it enters the capillary beds of the tissues, it encounters falling pO₂ which starts the conversion of some Hb-R to Hb-T. When a hemoglobin molecule snaps to the tight form, its four heme groups retract and the remaining oxygen gets ejected making it available for tissue metabolism. The transition from Hb-R to Hb-T is strongly enhanced by H-+ ions entering the red cells. A single H-+ ion can trigger the conversion of a molecule of hemoglobin to the tight form and eject its remaining oxygen. This is the mechanism for the “Bohr” effect.

Besides H-+ ions, increasing concentrations of Cl ions, CO₂ and DPG-4 all favor Hb-T. Most of the agents that drive hemoglobin to the tight form also raise the equilibrium constant for the first oxygen bound, which exaggerates the “s” shape of the dissociation curve and increases its maximum slop (Hill's coefficient”). −4 The release of oxygen is enhanced by 2,3-Diphosphoglycerate (DPG). There is about one molecule of DPG for every molecule of hemoglobin in red cells. More DPG both decreases the hemoglobin affinity for oxygen and steepens the slope of the “s-shaped” dissociation curve. Taken together, the effect is to force hemoglobin to release even more oxygen to tissues as pO 2 falls. It turns out that DPG plays a direct role in tightening the hemoglobin molecule and forcing its conversion to Hb-T.

Agents driving the hemoglobin the other way towards Hb-R are, besides oxygen, carbon monoxide (CO) and nitric oxide (NO). Hemoglobin has even more affinity for these gases than for oxygen. A heavy smoker may lower the O₂ concentration in the arterial blood by 20%. Increasing temperature favors Hb-R and decreases the maximum slope of the dissociation curve.

### Therapeutic Ozone Agent

The therapeutic ozone agent according to the invention may be administered in a form selected from the group consisting of a gas, a liquid formulation, a combination with O₂, and combinations thereof. The therapeutic ozone agent may be, for example, ozone gas, an ozone/oxygen gas mixture, an ozonated liquid, or ozone treated blood and/or blood products.

The therapeutic ozone agent may be administered, for example, in one of four ways over a period of time sufficient to diminish CO levels in the plasma, and, thusly, diminish the threat of CO bonding to hemoglobin. CO already bonded to hemoglobin receptors will, in some measure, be converted to CO₂, and then said CO₂ will then be exhaled from the body through the lungs. Removal of CO from blood plasma will reduce the threat of CO bonded hemoglobin, and to the extent that CO is converted to CO₂ on the hemoglobin receptor; the hemoglobin will again be free to carry oxygen.

Exemplary methods of therapeutic ozone agent (e.g., ozone or ozone/oxygen) delivery include:
The therapeutic ozone agent may be administered to a patient by the extra corporeal treatment of the patient's own blood at a dose of, for example, 5 mg. The resultant mixture may be re-infused by means of intravenous drip. For example, in autohemotherapy, about 50 to 100 ml of the patient's blood is withdrawn and exposed to the therapeutic ozone agent in a pre-determined concentration, then returned via intravenous catheter to the patient. In an exemplary embodiment, the quantity of blood exposed to the therapeutic ozone agent is, for example, 1 ml, 2, ml, 3, ml, 4 ml, 5 ml, 6 ml, 7 ml, 8 ml, 9 ml, 10 ml, 20 ml, 25 ml, 30 ml, 35 ml, 40 ml, 50 ml, 60 ml, 70 ml, 80 ml, 90 ml, or 100 ml. In an exemplary embodiment, blood is treated with the therapeutic ozone agent at concentrations of, for example, about 0.00001, 0.000025, 0.000050, 0.0001, 0.00025, 0.00050, 0.001, 0.0025, 0.0050, 0.01, 0.025, 0.050, 0.1, 0.25, 0.5, 1, 2, 3, 4, 5, 10, 20, 30, 40, 50, 60, 70, 80, 90, or 100 milligrams ozone therapeutic agent/ml of blood. In a preferred embodiment, the dosage is 20-50 micrograms/ml. In an exemplary embodiment the blood is exposed to the therapeutic ozone agent at a dosage of for example, about 0.00001, 0.000025, 0.000050, 0.0001, 0.00025, 0.00050, 0.001, 0.0025, 0.0050, 0.01, 0.025, 0.050, 0.1, 0.25, 0.5, 1, 2, 3, 4, 5, 6, 7, 8, 9, 10, 15, 20, or 25 mg of pure ozone, or a mix of ozone/oxygen at a concentration of, for example about 1%, 2%, 3%, 4%, 5%, 6%, 7%, 8%, 9%, or 10% ozone/oxygen. In exemplary embodiments, the blood is exposed to the therapeutic ozone agent for a time selected from, for example, about 1, 2, 3, 4, 5, 6, 7, 8, 9, 10, 15, 20, 25, 30, 35, 40, 45, 50, 55, or 60 minutes;
A dose of therapeutic ozone agent may administered by means of syringe inter muscularly. This method may not make contact with CO in the blood plasma or CO bonded to hemoglobin. In an exemplary embodiment, the therapeutic agent may be administered for a time selected from about 1, 2, 3, 4, 5, 6, 7, 8, 9, 10, 15, 20, 25, 30, 35, 40, 45, 50, 55, or 60 minutes, and ata dosage of for example, about 0.00001, 0.000025, 0.000050, 0.0001, 0.00025, 0.00050, 0.001, 0.0025, 0.0050, 0.01, 0.025, 0.050, 0.1, 0.25, 0.5, 1, 2, 3, 4, 5, 6, 7, 8, 9, 10, 15, 20, or 25 mg of pure ozone, or a mix of ozone/oxygen at a concentration of, for example about 1%, 2%, 3%, 4%, 5%, 6%, 7%, 8%, 9%, or 10% ozone/oxygen;
A dose of therapeutic ozone agent may be administered by means of an extra corporeal perfusion system constructed of material able to withstand oxidation. In an exemplary embodiment, the therapeutic agent may be administered for a time selected from about 1, 2, 3, 4, 5, 6, 7, 8, 9, 10, 15, 20, 25, 30, 35, 40, 45, 50, 55, or 60 minutes, and at a dosage of for example, about 0.00001, 0.000025, 0.000050, 0.0001, 0.00025, 0.00050, 0.001, 0.0025, 0.0050, 0.01, 0.025, 0.050, 0.1, 0.25, 0.5, 1, 2, 3, 4, 5, 6, 7, 8, 9, 10, 15, 20, 25 mg of pure ozone, or a mix of ozone/oxygen at a concentration of, for example, about 1%, 2%, 3%, 4%, 5%, 6%, 7%, 8%, 9%, or 10% ozone/oxygen;
The dose of therapeutic ozone agent may be administered by means of a lubricated Teflon rectal tube inserted into the rectum beyond the sigmoid curve into the colon. This method is preferred for because it is the simplest method of administration and does not rely on the already compromised veins of hospitalized persons. The introduction of the ozone therapeutic agent directly into the colon through a rectal tube inserted into the anus, around the sigmoid curve, and into the colon can overcome the inefficient or impossible introduction of oxygen through, for example, the nasal passages by means of a two-pronged nasal tube that usually is found to have fallen out of the nostrils, or by means of tracheal intubation. Empirically, hyperbaric chambers can get about a 2% increase in pO₂ (oxygen partial pressure); in contrast the methods and agents of the invention can get a circa 30% rise in PO₂ with no increase in methemoglobin. The 2% increase in pO₂ is not sufficient for a proper therapeutic benefit; the ability to get a rise in pO₂ from between the range of 2% to 30% will enable the therapeutic benefits as described herein, such as, for example, improving self-renewal, growth, mobilization, proliferation and/or differentiation of a therapeutic cell composition. In an exemplary embodiment, the therapeutic ozone agent may cause a rise in pO₂ of about 0.25%, 0.5%, 1%, 2%, 3%, 4%, 5%, 6%, 7%, 8%, 9%, 10%, 15%, 20%, 25%, 30%, or 35%. For example, see Example 1. In an exemplary embodiment, the therapeutic ozone agent may be administered by rectal insufflation for a time selected from 1, 2, 3, 4, 5, 6, 7, 8, 9, 10, 15, 20, 25, 30, 35, 40, 45, 50, 55, or 60 minutes, and at a dosage of, for example, about 0.00001, 0.000025, 0.000050, 0.0001, 0.00025, 0.00050, 0.001, 0.0025, 0.0050, 0.01, 0.025, 0.050, 0.1, 0.25, 0.5, 1, 2, 3, 4, 5, 6, 7, 8, 9, 10, 15, 20, 25, 30, 35, 40, 45, 50, 55, or 60 mg of pure ozone, or a mix of ozone/oxygen at a concentration of 1%, 2%, 3%, 3.7%, 4%, 5%, 6%, 7%, 8%, 9%, or 10% ozone/oxygen. In a preferred embodiment, ozone/oxygen may be obtained at a concentration of 50 mg ozone/L oxygen (ca. 3.7%). In an exemplary embodiment, ozone may be administered daily with 50 ml up to 500 ml, at concentrations of 25 micrograms/ml, 50 micrograms/ml, 75 micrograms/ml, or 100 micrograms/ml ozone/oxygen. In an exemplary embodiment, ozone may be administered daily (e.g., 5 days per week) for 1, 2, 3, 4, 5, or 6 weeks;

Based on the major role blood deoxygenation and hypoxia play in the onset and persistence of painful sickle cell crisis, and considering the established therapeutic properties of the therapeutic ozone agent, and the absence of negative side effects, an evaluation of the possible effectiveness of this treatment for the prevention and/or the timely resolution of the crisis was made, by means of controlled in vitro and clinical trials. It was encouraged by our extensive successful practice in numerous Havana City Hospitals, fundamentally that regarding the treatment of patients suffering circulatory insufficiencies, diabetes and many other diseases related to insufficient supply of oxygen to tissue. (Proceedings of the First Iberolatinamerican Congress on Ozone Applications. La Habana, 31 Oct.-3 Nov. 1990).

An ozone delivery system utilized herein delivers a measured amount of, for example, an ozone/oxygen admixture, and is able to measure, control, report and differentiate between the delivered-ozone and absorbed-dose of ozone. The system provides a controllable, measurable, accurate and reproducible amount of ozone that is delivered to a controllable, measurable, accurate and reproducible amount of a biological fluid and controls the rate of ozone absorption by the fluid resulting in a quantifiable absorbed-dose of ozone used in treatment.

The ozone delivery system may accomplish this by using a manufacturing component, control components, measuring components, a reporting component and calculating component (such as an ozone generator, gas flow meter, fluid pump, variable pitch platform, data acquisition device, inlet ozone concentration monitor, and exit ozone concentration monitor) that cooperate to manufacture and deliver a measured, controlled, accurate and reproducible amount of ozone (i.e., the delivered-ozone) to a fluid through the use of one or more gas-fluid contacting devices that provide for the interface between the ozone/oxygen admixture and fluid.

Using control components, measuring components, a reporting component and calculating components (such as a gas flow meter, fluid pump, variable pitch platform, data acquisition device, inlet ozone concentration monitor, and exit ozone concentration monitor) that cooperate, the system may instantly differentiate the delivered-ozone from the absorbed-dose of ozone.

An ozone delivery system particularly suitable to the present invention may include an ozone generator for the manufacture and control of a measured amount of an ozone/oxygen admixture and where the admixture volume contains the delivered-ozone. A commercially available ozone generator capable of producing ozone in a concentration range between 10 and 3,000,000 ppmv of ozone in an ozone/oxygen admixture may be employed. Ozone/oxygen admixture concentrations entering the gas-fluid contacting device are instantly and constantly measured in real time, through an inlet ozone concentration monitor that may utilize UV absorption as a detection methodology. A flow meter controls and measures the delivery of the delivered-ozone in an ozone/oxygen admixture to the gas-fluid contacting device at a specified admixture flow rate. Ozone/oxygen admixture flow rates are typically in the range between 0.1 and 5.0 liters per minute.

The concentration of the ozone/oxygen admixtures exiting the gas-fluid contacting device and where the admixture volume contains the residual-ozone, are instantly and constantly measured in real time through an exit ozone concentration monitor that may utilize UV absorption as a detection methodology.

The therapeutic ozone agent of the invention may be administered, for example, in single or divided doses of one, two, three, or four times daily. It may be advisable to start a patient on a low dose to a high dose.

The therapeutic ozone agent as described herein may be administered, for example, for one day, two days, three days, four days, five days, six days, a week, two weeks, three weeks, four weeks, five weeks, six weeks, a month, two months, three months, four months, or more. The therapeutic ozone agent as described herein may be administered, for example, for a prolonged period, that is, for as long as the potential for a disease or condition remains and/or the symptoms continue.

The therapeutic ozone agent can be administered to the patient on a regimen of, for example, one, two, three, four, five, six, or other doses per day. The therapeutic ozone agent as described herein may be administered, for example, every day, every two days, or every three days.

Similarly, the length of time of administration of the therapeutic ozone agent may vary. In particular embodiments, a therapeutic ozone agent of the present invention may be provided for about or at least 30 seconds, 1 minute, 2 minutes, 3 minutes, 5 minutes, 10 minutes, 15 minutes, 30 minutes, 1 hour, 2 hours, 3 hours, four hours five hours, six hours, eight hours, twelve hours, twenty-four hours, or greater than twenty-four hours. A therapeutic ozone agent of the present invention may be administered, for example, in a single dose or multiple doses, with varying amounts of time between administered doses.

Present ozone generators have basic means for controlling the concentration and delivery of ozone gas. Oxygen is generally passed through a machine containing permanent electrodes; the gas chambers of present generators are often permanent as well. Some generators include components that neutralize excess ozone. Other generators continuously vent ozone. Present ozone generators often include components for gas containment or pass oxygen through reaction chambers that are permanent and reusable, lending to sterility issues. Medical professionals often inject the gas through a bacterial filter to address such sterility issues.

The following patent publications illustrate and describe various background apparatuses, methods and/or systems related to generating ozone. US Patent Publication No. 2005/0074501 (Murphy et al.) teaches an apparatus, in an embodiment, including an ozone generator connected to a scavenger and an ozone administrator via network of tubing and valves. US Patent Publication No 2007/002,5890 (Joshi et al.) teaches an apparatus that in various embodiments includes a syringe having a barrel and a plunger and having an ozone generator associated therewith. US Patent Publication No. 2003/0165411 (Engelhard) teaches an ozone generator that is a module having a threaded shaft serving as an electrode and which mechanically secures the various elements with one another. U.S. Pat. No. 6,270,733 (Rodden) teaches a tubular ozone generator comprising concentric inner tubular electrode/dielectric with inner electrode and outer tubular electrode with corona discharge zone between the inner tubular electrode/dielectric and outer tubular electrode. U.S. Pat. No. 6,110,431 (Dunder) teaches an ozone dispensing system comprising an ozone gas generating means, electrical means to control the concentration of ozone produced by said ozone gas generating means, means to control the concentration of ozone in preset dispensed volume, an oxygen supply and venting means disposed between said ozone gas generating means and said dispensing of said ozone, said venting means for continuous venting of said ozone. U.S. Pat. No. 5,052,382 (Wainwright) teaches an apparatus for the controlled generation and administration of ozone, which apparatus comprises a generator for generating ozone, a monitor for monitoring the ozone, a dosage device for providing a correct amount of ozone for administration, and a computer control device for controlling the operation of at least one of the generator, the monitor and the dosage device.

Therapeutic ozone agents of the invention may be used in the treatment of acute and chronic conditions, including, but not limited to, hospitalized patients, cardiovascular operations, chronic anemia, anemia following major surgery, coronary infarction and associated problems, chronic pulmonary disease, cardiovascular patients, autologous transfusions, as an enhancement to packed red blood cells transfusion (hemorrhage, traumatic injury, or surgery) congestive heart failure, myocardial infarction (heart attack), stroke, peripheral vascular disease, intermittent claudication, circulatory shock, hemorrhagic shock, anemia and chronic hypoxia, respiratory alkalemia, metabolic alkalosis, sickle cell anemia, reduced lung capacity caused by pneumonia, surgery, complications associated with angioplasty, pneumonia, trauma, chest puncture, gangrene, anaerobic infections, blood vessel diseases such as diabetes, substitute or complement to treatment with hyperbaric pressure chambers, intra-operative red cell salvage, cardiac inadequacy, anoxia-secondary to chronic indication, organ transplant, carbon monoxide, nitric oxide, and cyanide poisoning.

This invention is related to a method of treating a subject for, for example, any one or more of the diseases listed herein, comprising the steps of administration of an ozone therapeutic agent of the invention via insufflation, or treating red blood cells or whole blood ex vivo with one or more compositions of the present invention, followed by administering the prepared red blood cells or whole blood to said subject.

Because the compounds, compositions, and methods of the present invention are capable of allosterically modifying hemoglobin to favor the low oxygen affinity “T” state (i.e., right shifting the equilibrium curve), RBC's or whole blood treated with the compounds of the present invention and subsequently purified will be useful in treating a variety of disease states in mammals, including humans, wherein tissues suffer from low oxygen tension, such as cancer and ischemia. RBC's or whole blood treated with the compounds of the present invention and subsequently purified may be administered to patients in whom the affinity of hemoglobin for oxygen is abnormally high. For example, certain hemoglobinopathies, certain respiratory distress syndromes, e.g., respiratory distress syndromes in new born infants aggravated by high fetal hemoglobin levels, and conditions in which the availability of hemoglobin/oxygen to the tissues is decreased (e.g., in ischemic conditions such as peripheral vascular disease, coronary occlusion, cerebral vascular accidents, or tissue transplant). The compounds and compositions may also be used to inhibit platelet aggregation, antithrombotic purposes, and wound healing.

Additionally, the compounds and compositions of the present invention can be added to whole blood or packed cells preferably at the time of storage or at the time of transfusion in order to facilitate the dissociation of oxygen from hemoglobin and improve the oxygen delivering capability of the blood. When blood is stored, the hemoglobin in the blood tends to increase its affinity for oxygen by losing 2,3-diphosphoglycerides. As described above, the compounds and compositions of this invention are capable of reversing and/or preventing the functional abnormality of hemoglobin observed when whole blood or packed cells are stored. The compounds and compositions may be added to whole blood or red blood cell fractions in a closed system using an appropriate reservoir in which the compound or composition is placed prior to storage or which is present in the anticoagulating solution in the blood collecting bag.

Administration to a patient can be achieved by intravenous or intraperitoneal injection where the dose of treated red blood cells or whole blood and the dosing regiment is varied according to individual's sensitivity and the type of disease state being treated. Solid tumors are oxygen deficient masses. The compounds, compositions and methods of this invention may be exploited to cause more oxygen to be delivered to tumors, increasing radical formation and thereby increasing tumor killing during radiation. In this context, such ozone-treated blood will only be used in conjunction with radiotherapy.

The compounds, compositions and methods of this invention may be exploited to cause more oxygen to be delivered at low blood flow and low temperatures, providing the ability to decrease or prevent the cellular damage, e.g., myocardial or neuronal, typically associated with these conditions.

The compounds, compositions and methods of this invention may be exploited to decrease the number of red blood cells required for treating hemorrhagic shock by increasing the efficiency with which they deliver oxygen.

Damaged tissues heal faster when there is better blood flow and increased oxygen tension. Therefore, the compounds, compositions and methods of this invention may be exploited to speed wound healing. Furthermore, by increasing oxygen delivery to wounded tissue, the compounds, compositions and methods of this invention may play a role in the destruction of infection causing bacteria at a wound.

### Improving Therapeutic Cell Activity

In specific embodiments, the present invention is directed to improving self-renewal, growth, mobilization, proliferation and/or differentiation of a therapeutic cell composition, including methods for promoting regenerative cell growth, such as for example, therapeutic cell growth in vivo, by administering an ozone therapeutic agent, in accordance with the methods of the invention. Without being bound by theory, the high PO₂ (oxygen partial pressure) ensuing from the systemic introduction of the ozone therapeutic agent will improve general tissue function, health, longevity, and general systemic well-being that, for example, hyperbaric oxygen chambers cannot provide and that the introduction of oxygen through the respiratory system (nostrils and lungs) cannot provide.

The therapeutic or regenerative cells may be comprised of, e.g., stem cells, progenitor cells or combinations thereof. In certain embodiments, administration of multiple doses of therapeutic or regenerative cells may be needed to derive a therapeutic benefit. In addition, additives such as one or more growth factors may be administered with the therapeutic or regenerative cells. In a preferred embodiment, the therapeutic or regenerative cells are administered with angiogenic growth factors alone or in combination with other additives. The therapeutic or regenerative cells may also be administered with one or more immunosuppressive drugs. In certain embodiments, administration of multiple doses of the therapeutic ozone agent may be used.

### Therapeutic Cells

As used herein, the term “therapeutic cell composition” refers to a cell composition in a biologically acceptable carrier for administration to a patient, wherein the cells may include, for example, stem cells, progenitor cell, embryonic stem cells, pluripotent stem cells, adult stem cells, induced pluripotent stem cells, genetically engineered cells, and combinations thereof.

As used herein, “stem cell” refers to a multipotent regenerative cell with the potential to differentiate into a variety of other cell types, which perform one or more specific functions and have the ability to self-renew. Some of the stem cells disclosed herein may be multipotent. The term “stem cell” generally refers to a cell that on division faces two developmental options: the daughter cells can be identical to the original cell (self-renewal) or they may be the progenitors of more specialized cell types (differentiation). The stem cell is therefore capable of adopting one or other pathway (a further pathway exists in which one of each cell type can be formed). Stem cells are therefore cells which are not terminally differentiated and are able to produce cells of other types.

The term “xenogeneic cell” refers to a cell that derives from a different animal species than the animal species that becomes the recipient animal host in a transplantation or vaccination procedure.

The term “allogeneic cell” refers to a cell that is of the same animal species but genetically different in one or more genetic loci as the animal that becomes the “recipient host”. This usually applies to cells transplanted from one animal to another non-identical animal of the same species. The term “syngeneic cell” refers to a cell which is of the same animal species and has the same genetic composition for most genotypic and phenotypic markers as the animal who becomes the recipient host of that cell line in a transplantation or vaccination procedure. This usually applies to cells transplanted from identical twins or may be applied to cells transplanted between highly inbred animals.

“Bone marrow derived progenitor cell” (BMDC) or “bone marrow derived stem cell” refers to a primitive stem cell with the machinery for self-renewal constitutively active. Included in this definition are stem cells that are totipotent, pluripotent and precursors.

A “precursor cell” can be any cell in a cell differentiation pathway that is capable of differentiating into a more mature cell. As such, the term “precursor cell population” refers to a group of cells capable of developing into a more mature cell. A precursor cell population can comprise cells that are totipotent, cells that are pluripotent and cells that are stem cell lineage restricted (i.e. cells capable of developing into less than all hematopoietic lineages, or into, for example, only cells of erythroid lineage).

As used herein, the term “totipotent cell” refers to a cell capable of developing into all lineages of cells. Similarly, the term “totipotent population of cells” refers to a composition of cells capable of developing into all lineages of cells.

Bone marrow derived stem cells contain two well-characterized types of stem cells. Mesenchymal stem cells (MSC) normally form chondrocytes and osteoblasts. Hematopoietic stem cells (HSC) are of mesodermal origin that normally give rise to cells of the blood and immune system (e.g., erythroid, granulocyte/macrophage, magakaryocite and lymphoid lineages). In addition, hematopoietic stem cells also have been shown to have the potential to differentiate into the cells of the liver (including hepatocytes, bile duct cells), lung, kidney (e.g., renal tubular epithelial cells and renal parenchyma), gastrointestinal tract, skeletal muscle fibers, astrocytes of the CNS, Purkinje neurons, cardiac muscle (e.g., cardiomyocytes), endothelium and skin.

As used herein, “progenitor cell” refers to a multipotent regenerative cell with the potential to differentiate into more than one cell type and has limited or no ability to self-renew. “Progenitor cell”, as used herein, also refers to a unipotent cell with the potential to differentiate into only a single cell type, which performs one or more specific functions and has limited or no ability to self-renew. In particular, as used herein, “endothelial progenitor cell” refers to a multipotent or unipotent cell with the potential to differentiate into vascular endothelial cells.

As used herein, “precursor cell” refers to a unipotent regenerative cell with the potential to differentiate into one cell type. Precursor cells and their progeny may retain extensive proliferative capacity, e.g., lymphocytes and endothelial cells, which can proliferate under appropriate conditions.

Embryonic Stem (ESCs) cells may be isolated from the inner cell mass (ICM) of the blastocyst, which is the stage of embryonic development when implantation occurs. Pluripotent stem cells are true stem cells, with the potential to make any differentiated cell in the body. However, they cannot contribute to making the extraembryonic membranes which are derived from the trophoblast. Several types of pluripotent stem cells have been found. Multipotent stem cells are true stem cells but can only differentiate into a limited number of types. For example, the bone marrow contains multipotent stem cells that give rise to all the cells of the blood but not to other types of cells. Multipotent stem cells are found in adult animals. It is thought that every organ in the body contains them where they can replace dead or damaged cells. Also as used herein, the term “pluripotent cell” refers to a cell capable of developing into a variety (albeit not all) lineages and are at least able to develop into all hematopoietic lineages (e.g., lymphoid, erythroid, and thrombocytic lineages).

Methods of characterizing stem cells are known in the art, and include the use of standard assay methods such as clonal assay, flow cytometry, long-term culture and molecular biological techniques e.g. PCR, RT-PCR and Southern blotting.

Adult stem cells comprise a wide variety of types including neuronal, skin and the blood forming stem cells which are the active component in bone marrow transplantation.

These latter stem cell types are also the principal feature of umbilical cord-derived stem cells. Adult stem cells can mature both in the laboratory and in the body into functional, more specialized cell types although the exact number of cell types is limited by the type of stem cell chosen.

Induced pluripotent stem cells, commonly abbreviated as iPS cells or iPSCs, are a type of pluripotent stem cell artificially derived from a non-pluripotent cell, typically an adult somatic cell, by inserting certain genes. iPS cells are reviewed and discussed in Takahashi, K. & Yamanaka (2006), Yamanaka S, et. al. (2007), Wemig M, et. al. (2007), Maherali N, et. al. (2007), Yu J, et al. (2007) and Takahashi et al., (2007), all of which are incorporated herein by reference. iPS cells are typically derived by transfection of certain stem cell-associated genes into non-pluripotent cells, such as adult fibroblasts. Transfection is typically achieved through viral vectors, for example through retroviral reprogramming Transfected genes include the master transcriptional regulators Oct-3/4 (Pouf51) and Sox2, although it is suggested that other genes enhance the efficiency of induction. After 3-4 weeks, small numbers of transfected cells begin to become morphologically and biochemically similar to pluripotent stem cells, and are typically isolated through morphological selection, doubling time, or through a reporter gene and antibiotic infection. IPSCs may be induced from somatic cells such as fibroblasts by transfection with one or more transcription factors. In some cases, cells are transformed with Oct3/4, Sox2, c-Myc and Klf4. The cells may be additionally transfected with other genes, including transcription factors and/or marker genes. The genes may be introduced using a transposon system such as the Cre/IoxP recombination system, or using non-integrating vectors in order to produce iPSCs free of exogenous reprogramming genes. Transfection may be achieved using viral vectors, such as a retrovirus. The virus may be an amphotropic virus. Once the cells have been transfected, they may be grown on feeder cells before transfer to an ESC culture medium.

iPS cells may be derived from any suitable cell type, including lung, foreskin fibroblasts, skin fibroblasts, keratinocytes, blood progenitor cells, bone marrow cells, hepatocytes, gastric epithelial cells, pancreatic cells, neural stem cells, B lymphocytes, ES derived somatic cells and embryonic fibroblasts. In some cases, the cells are not human dermal fibroblasts. The IPSCs may exhibit similar patterns of gene expression and phenotype to ESCs.

### Sources of Induced Pluripotent Stem Cells

Several methods have now been provided for the isolation of pluripotent stem cells that do not lead to the destruction of an embryo, e.g. by transforming (inducing) adult somatic cells or germ cells. These methods may include: 1. Reprogramming by nuclear transfer. This technique involves the transfer of a nucleus from a somatic cell into an oocyte or zygote. In some situations this may lead to the creation of an animal-human hybrid cell. For example, cells may be created by the fusion of a human somatic cell with an animal oocyte or zygote or fusion of a human oocyte or zygote with an animal somatic cell; 2. Reprogramming by fusion with embryonic stem cells. This technique involves the fusion of a somatic cell with an embryonic stem cell. This technique may also lead to the creation of animal-human hybrid cells, as in 1 above; 3. Spontaneous re-programming by culture. This technique involves the generation of pluripotent cells from non-pluripotent cells after long term culture; 4. Reprogramming by defined factors. For example the generation of iPS cells by the retrovirus-mediated introduction of transcription factors. Methods 1-4 are described and discussed by Shinya Yamanaka in Strategies and New Developments in the Generation of Patient-Specific Pluripotent Stem Cells (Cell Stem Cell 1, July 2007 Elsevier Inc), incorporated herein by reference. 5. Derivation of hESC lines from single blastomeres or biopsied blastomeres; 6. hESC lines obtained from arrested embryos which stopped cleavage and failed to develop to morula and blastocysts in vitro; 7. Parthogenesis (or Parthenogenesis). This technique involves chemical or electrical stimulation of an unfertilised egg so as to cause it to develop into a blastomere from which embryonic stem cells may be derived; 8. Stem cells of fetal origin. These cells lie between embryonic and adult stem cells in terms of potentiality and may be used to derive pluripotent or multipotent cells.

Induced pluripotent stem cells have the advantage that they can be obtained by a method that does not cause the destruction of an embryo, more particularly by a method that does not cause the destruction of a human or mammalian embryo. As such, aspects of the invention may be performed or put into practice by using cells that have not been prepared exclusively by a method which necessarily involves the destruction of human or animal embryos from which those cells may be derived.

### Mesenchymal Stem Cells

Mesenchymal stem cells are known as being multipotent and exhibit the potential for differentiation into different cells/tissue lineages, including cartilage, bone, adipose tissue, tendon, and ligament. These multipotent mesenchymal progenitor cells are denoted as stromal or mesenchymal stem cells. Bone marrow contains two main cell types: hematopoietic cells and stromal cells. The stem cells for non-hematopoietic tissues are referred as mesenchymal cells because of their ability to differentiate as mesenchymal or stromal cells.

Accordingly, in this specification mesenchymal stem cells (MSCs) refers to multipotent stem cells capable of differentiation into osteoblasts, chondrocytes, myocytes, adipocytes and endothelium. In this specification MSCs particularly refers to multipotent stem cells capable of differentiation into osteoblasts as part of the process of formation of bone.

Mesenchymal cells are easily obtainable from bone marrow by minimally invasive techniques and can be expanded in culture and permitted to differentiate into the desired lineage. Differentiation can be induced by the application of specific growth factors. The transforming growth factor beta (TGF-beta) superfamily member proteins such as the bone morphogenetic proteins (BMPs) are important factors of chondrogenic and osteogenic differentiation of mesenchymal stem cells.

Suitable MSCs may be obtained or derived from bone marrow mononuclear cells (BMMNCs) collected from aspirates of bone marrow (e.g. Wexler et al. Adult bone marrow is a rich source of human mesenchymal ‘stem’ cells but umbilical cord and mobilized adult blood are not. HAEMOPOIESIS AND LEUCOCYTES British Journal of Haematology 121(2):368-374, April 2003.) or Wharton's Jelly of the umbilical cord (e.g. Ta et al. Long-term Expansion and Pluripotent Marker Array Analysis of Wharton's Jelly-Derived Mesenchymal Stem Cells. Stem Cells Dev. 2009 Jul. 20 (Epub)).

### Culture of Stem Cells

Any suitable method of culturing stem cells may be used, and any suitable container may be used to propagate stem cells. Suitable containers include those described in US Patent Publication US 2007/0264713 (Terstegge).

Containers may include bioreactors and spinners, for example. A “bioreactor” is a container suitable for the cultivation of eukaryotic cells, for example animal cells or mammalian cells, such as in a large scale. A typical cultivation volume of a regulated bioreactor is between 20 ml and 500 ml. The bioreactor may comprise a regulated bioreactor, in which one or more conditions may be controlled or monitored, for example, oxygen partial pressure. Devices for measuring and regulating these conditions are known in the art. For example, oxygen electrodes may be used for oxygen partial pressure. The oxygen partial pressure can be regulated via the amount and the composition of the selected gas mixture (e.g., air or a mixture of air and/or oxygen and/or nitrogen and/or carbon dioxide). Suitable devices for measuring and regulating the oxygen partial pressure are described by Bailey, J E. (Bailey, J E., Biochemical Engineering Fundamentals, second edition, McGraw-Hill, Inc. ISBN 0-07-003212-2 Higher Education, (1986)) or Jackson A T. Jackson A T., Verfahrenstechnik in der Biotechnologie, Springer, ISBN 3540561900 (1993)).

Other suitable containers include spinners. Spinners are regulated or unregulated bioreactors, which can be agitated using various agitator mechanisms, such as glass ball agitators, impeller agitators, and other suitable agitators. The cultivation volume of a spinner is typically between 20 ml and 500 ml. Roller bottles are round cell culture flasks made of plastic or glass having a culture area of between 400 and 2000 cm². The cells are cultivated along the entire inner surface of these flasks; the cells are coated with culture medium accomplished by a “rolling” motion, i.e. rotating the bottles about their own individual axis.

Alternatively, culture may be static, i.e. where active agitation of the culture/culture media is not employed. By reducing agitation of the culture, aggregates of cells may be allowed to form. Whilst some agitation may be employed to encourage distribution and flow of the culture media over the cultured cells this may be applied so as not to substantially disrupt aggregate formation. For example, a low rpm agitation, e.g. less than 30 rpm or less than 20 rpm, may be employed.

Propagation with Passage

Methods of cell culture may comprise passaging, or splitting during culture. The methods may involve continuous or continual passage.

Cells in culture may be dissociated from the substrate or flask, and “split”, subcultured or passaged, by dilution into tissue culture medium and replating/re-cultuting.

The term “passage” may generally refer to the process of taking an aliquot of a cell culture, dissociating the cells completely or partially, diluting and inoculating into medium. The passaging may be repeated one or more times. The aliquot may comprise the whole or a portion of the cell culture. The cells of the aliquot may be completely, partially or not confluent. The passaging may comprise at least some of the following sequence of steps: aspiration, rinsing, trypsinization, incubation, dislodging, quenching, re-seeding and aliquoting.

The cells may be dissociated by any suitable means, such as mechanical or enzymatic means known in the art. The cells may be broken up by mechanical dissociation, for example using a cell scraper or pipette. The cells may be dissociated by sieving through a suitable sieve size, such as through 100 micron or 500 micron sieves. The cells may be split by enzymatic dissociation, for example by treatment with collagenase or trypLE harvested. The dissociation may be complete or partial.

The dilution may be of any suitable dilution. The cells in the cell culture may be split at any suitable ratio. For example, the cells may be split at a ratio of 1:2 or more, 1:3 or more, 1:4 or more or 1:5 or more. Thus, stem cells may be passaged for 1 passage or more. For example, stem cells may be passaged for 2, 3, 4, 5, 6, 7, 8, 9, 10, 11, 12, 13, 14, 15, 16, 17, 18, 19, 20, 21, 22, 23, 24, 25 passages or more. Passages may be expressed as generations of cell growth. Stem cells may be propagated for 1, 2, 3, 4, 5, 6, 7, 8, 9, 10, 11, 12, 13, 14, 15, 16, 17, 18, 19, 20, 21, 22, 23, 24, 25 generations or more. Stem cells may be propagated for 1, 2, 3, 4, 5, 6, 7, 8, 9, 10, 11, 12, 13, 14, 15, 16, 17, 18, 19, 20, 21, 22, 23, 24, 25 cell doublings or more

### Co-Culture and Feeders

Methods may comprise culturing stem cells in the presence or absence of co-culture. The term “co-culture” refers to a mixture of two or more different kinds of cells that are grown together. The two or more different kinds of cells may be grown on the same surfaces, such as particles or cell container surfaces, or on different surfaces. The different kinds of cells may be grown on different particles.

Feeder cells may mean cells which are used for or required for cultivation of cells of a different type. In the context of stem cell culture, feeder cells have the function of securing the survival, proliferation, and maintenance of cell pluripotency or multipotency. Cell pluripotency/multipotency may be ensured by directly co-cultivating the feeder cells. For example, the inner surface of the container such as a culture dish may be coated with a feeder cell layer. The feeder cells release nutrients into the culture medium. Alternatively, or in addition, the feeder cells may be cultured in a medium to condition it. The conditioned medium may be used to culture the stem cells. Thus, arrangements in which feeder cells are absent or not required are also possible.

### Gene Therapy

In some preferred embodiments, the therapeutic cell composition may comprise therapeutic genes for delivery into the body, such as for example, the heart. In some embodiments, the therapeutic gene is a transgene. For example, the delivery cells—e.g. the therapeutic cell are removed from the body, and a therapeutic transgene is introduced into them via vehicles well known to those skilled in the art such as those used in direct-gene-transfer methods. For example, while still in the laboratory, the genetically modified cells are tested and then allowed to grow and multiply and, finally, are infused back into the patient. Alternatively, allogeneic cells that bear normal, endogenous genes can reverse a deficiency in a particular target tissue. Use of cells bearing either transgenes or normal, endogenous genes is referred to herein as gene therapy.

### Administration of a Therapeutic Cell Composition

The introduction of the therapeutic cell composition of the invention can be accomplished by infusion of the cells either into a peripheral vein or a central veins. Direct injection into the pulmonary circulation can also be adopted, for example through a Swan Ganz catheter. Injection into the right ventricle or right atrium may be carried out using the pacing port of a Swan Ganz catheter. The infusion can be done either in a bolus form i.e. injection of all the cells during a short period of time, or it may be accomplished by a continuous infusion of small numbers of cells over a long period of time, or alternatively by administration of limited size boluses on several occasions over a period of time. Re-introduction of stem cells into the lungs can also be accomplished through inhalation of the cells using known pulmonary administration methods, such as an inhaler. The infusion may be done intravascularly and/or intramuscularly. Preferred infusion is intravascular, more preferably intra-arterial most preferably intra-coronary using a catheter coated with a biocompatible material. The intracoronary delivery may be in a single dose or multiple doses or a single dose on different days or a multiple dose on different days.

Administration of the therapeutic cell composition can be by any means known in the art, for example, by local injection, infusion, etc. Cells would be pretreated at optimal concentration most likely between pg/ml-1 microg/ml for a 10,000 cells (in 1 ml).

The routes of administration for the cells are known in the art and include injection into a subcutaneous, dermal or intramuscular in site distant from the injury or localized vascular delivery using a catheter based mechanism. The cells may be administered to, for example the patient's vasculature. The cells may also be administered via a scaffold, e.g., a resorbable scaffold, or a bandage known in the art.

Prior to administration to a patient, the cells may be grown in cell culture to, for example, promote differentiation towards a epithelial and/or endothelial phenotype. The cell culture may be performed on a scaffold material, e.g., a resorbable scaffold, to generate a two or three dimensional construct that can be placed on or within the patient. Prior to administration to a patient, the cells could also be modified by gene transfer such that expression of one or more genes, e.g., an angiogenic gene, in the modified regenerative cells is altered.

In one embodiment, direct administration of cells to the site of intended benefit is preferred. This may be achieved by intravenous injection. Routes of administration known to one of ordinary skill in the art, include but are not limited to, subcutaneous, dermal or intramuscular and may or may not include a catheter based mechanism of delivery. Cells may be injected in a single bolus, through a slow infusion, or through a staggered series of applications separated by several hours or, provided cells are appropriately stored, several days or weeks. In one embodiment, the route of delivery will include intravenous delivery through a standard peripheral intravenous catheter or a central venous catheter. The flow of cells may be controlled by serial inflation/deflation of distal and proximal balloons located within the patient's vasculature, thereby creating temporary no-flow zones which promote cellular engraftment or cellular therapeutic action. In another embodiment, cells may be resuspended in an artificial or natural medium or tissue scaffold prior to being administered to the patient. Systemic therapy would involve injection into the vascular system.

### Combination Active Agents

In addition, the therapeutic ozone agent, according to the invention, may be used in combination with, for example a calcineurin inhibitor, e.g. cyclosporin A or FK 506; a mTOR inhibitor, e.g. rapamycin, 40-O-(2-hydroxyethyl)-rapamycin, CCI779, ABT578. AP23573, AP23464, AP23675, AP23841, TAFA-93, biolimus-7 or biolimus-9; an ascomycin having immunosuppressive properties, e.g. ABT-281, ASM981, etc.; corticosteroids; cyclophosphamide; azathioprene; methotrexate: leflunomide; mizoribine; mycophenolic acid or salt; mycophenolate mofetil; 15-deoxyspergualine or an immunosuppressive homologue, analogue or derivative thereof; a PKC inhibitor, e.g. as disclosed in WO 02/38561 or WO 03/82859, e.g. the compound of Example 56 or 70; a JAK3 kinase inhibitor, e.g. N-benzyl-3,4-dihydroxy-benzylidene-cyanoacetamide.quadrature.-cyano-(3,4-dihydroxy)-]N-benzylcinnamamide (Tyrphostin AG 490), prodigiosin 25-C (PNU156804), [4-(4′-hydroxyphenyl)-amino-6,7-dimethoxyquinazoline] (WHI-P131), [4-(3′-bromo-4′-hydroxylphenyl)-amino-6,7-dimethoxyquinazoline] (WHI-P154), [4-(3′,5 ‘-dibromo-4’-hydroxylphenyl)-amino-6,7-dimethoxyquinazoline] WHI-P97, KRX-211, 3-{(3R,4R)-4-methyl-3-[methyl-(7H-pyrrolo [2,3-d]pyrimidin-4-yl)-amino]-pi-peridin-1-yl}-3-oxo-propionitrile, in free form or in a pharmaceutically acceptable salt form, e.g. mono-citrate (also called CP-690,550), or a compound as disclosed in WO 04/052359 or WO 05/066156; immunosuppressive monoclonal antibodies, e.g., monoclonal antibodies to leukocyte receptors, e.g., MHC, CD2, CD3, CD4, CD7, CD8. CD25, CD28, CD40, CD45, CD52, CD58, CD80, CD86 or their ligands; other immunomodulatory compounds, e.g. a recombinant binding molecule having at least a portion of the extracellular domain of CTLA4 or a mutant thereof, e.g. an at least extracellular portion of CTLA4 or a mutant thereof joined to a non-CTLA4 protein sequence, e.g. CTLA4Ig (for ex. designated ATCC 68629) or a mutant thereof, e.g. LEA29Y: adhesion molecule inhibitors, e.g. LFA-1 antagonists, ICAM-1 or −3 antagonists, VCAM-4 antagonists or VLA-4 antagonists; or a chemotherapeutic agent, e.g. paclitaxel, gemcitabine, cisplatinum, doxorubicin or 5-fluorouracil; or an anti-infectious agent Immunomodulatory drugs which are prone to be useful in combination with a compound of the present invention include e.g. mediators, e.g. inhibitors, of mTOR activity, including rapamycin and rapamycin derivatives, e.g. including 40-O-alkyl-rapamycin derivatives, such as 40-O-hydroxyalkyl-rapamycin derivatives, such as 40-O-(2-hydroxy)-ethyl-rapamycin (everolimus), 32-deoxo-rapamycin derivatives and 32-hydroxy-rapamycin derivatives, such as 32-deoxorapamycin, 16-O-substituted rapamycin derivatives such as 16-pent-2-ynyloxy-32-deoxorapamycin, 16-pent-2-ynyloxy-32 (S or R)-dihydro-rapamycin. 16-pent-2-ynyloxy-32(S or R)-dihydro-40-O-(2-hydroxyethyl)-rapamycin. rapamycin derivatives which are acylated at the oxygen group in position 40, e.g. 40-[3-hydroxy-2-(hydroxy-methyl)-2-methylpropanoate]-rapamycin (also known as CCI779), rapamycin derivatives which are substituted in 40 position by heterocyclyl. e.g. 40-epi-(tetrazolyl)-rapamycin (also known as ABT578), the so-called rapalogs. e.g. as disclosed in WO9802441, WO0114387 and WO0364383, such as AP23573, and/or compounds disclosed under the name TAFA-93 and/or biolimus (biolimus A9).

The therapeutic ozone agent, according to the invention, may be used in combination with Protein Kinase C Inhibitors and/or suppressors of T cell activation, in particular indolylmaleimide derivatives such as sotrastaurin (3-(1.H.-indol-3-yl)-4-[2-(4-methyl-piperazin-1-yl)-quinazolin-4-yl]-pyrr-ole-2,5-dione), for instance in order to inhibit the adaptive immune system and thereby further enhancing the therapeutic effects of IL-1beta compounds.

The therapeutic ozone agent, according to the invention, may be used in combination with anti-cytokines and/or anti-interleukins, in particular IL-17 binding compounds disclosed in WO2006/013107, optionally in combination with sotrastaurin.

The therapeutic ozone agent, according to the invention, may be used in combination with anti TNFalpha compounds such as etanercept, adalimumab, infliximab, for instance in the treatment of RA and other (auto)-inflammatory diseases.

Among the agents enhancing hemodynamics, anticoagulants, such as warfarin, acenocoumarol, phenprocoumon, phenindione, heparin, fondaparinux, idraparinux, hirudin, lepirudin, bivalirudin, argatroban, dabigatran, ximelagatran, batroxobin or hementin, blood flow enhancers, such as chlofluperol, trifluperol, droperidol, metaclopramide, loxapine or butoxamine, may be mentioned.

Especially preferred as combination partners are other co-agents for treatment of, for example, SCD, such as inducers of expression of fetal hemoglobin, e.g. hydroxyurea, 5-azacytidine, erythropoietine, butyrates, e.g. sodium butyrate or arginine butyrate, or acetates; inhibitors of hemoglobin polymerization (sickling), such as nitric oxide or drugs delivering nitric oxide; reducers of intracellular hemoglobin concentrations, e.g. K—Cl-contransporter inhibitors or Ca-dependent potassium channel inhibitors, e.g. clotrimauzole or seniapoc, or Magnesium salts. e.g. MG pidolate; or other drugs, e.g. Nicosan.

Also analgesics and narcotics are among the preferred combination partners, for example, NSAIDs such as salicylates, such as acetylsalicylic acid, diflunisal or salsalate, p-aminophenol derivatives, such as paracetamol or phenacetin, propionic acid derivatives, such as ibuprofen, naproxen, fenoprofen, ketoprofen, flurbiprofen, oxaprocin or loxoprofen, acetic acid derivatives, such as indomethacin, sulindac, etodolac, ketorolac, diclofenac or nabumetone, enolic acid (oxicam) derivatives, such as piroxicam, meloxicam, tenoxicam, droxicam, lornoxicam or isoxicam, fenamic acid derivatives, such as mefenamic acid, meclofenamic acid, flufenamic acid or tolfenamic acid, selective COX-2 inhibitors, such as celecoxib, rofecoxib, valdecoxib, parecoxib, lumiracoxib, etorixicib or firocoxib, sulphonanilides, such as nimesulide, or others, such as licofelone, flurpitine, opiates or opioids, such as morphine, dextromorphan, codeine, oxycodone, hydrocodone, dihydromorphine, pethidine buprenorphine, tramadol or venlafaxine, tricyclic antidepressants, such as amitriptyline, carbamazepine, gabapentin, pregabalin, psychotropic analgesic agents, e.g. cannabinoids, such as tetrahydrocannabinol, ketamine, clonidine or mexiletine, orphenadrine, cyclobenzaprine, scopolamine, atropine, gabapentin, methadone, ketobemidone, piritramide, or the like.

### Indications

The present invention relates to the use of therapeutic ozone agents, its use as a therapeutic agent, alone or in combination in medicine and for the prophylaxis and/or treatment of, for example, hypoxic conditions, cancer, autoimmune diseases, fibrotic diseases, inflammatory diseases, neurodegenerative diseases, infectious diseases, lung diseases, heart and vascular diseases and metabolic diseases. Also disclosed are pharmaceutical formulations preferably in form of a liquid solution containing the inventive therapeutic ozone agent.

This invention is related to a method of treating a subject for, for example, any one or more of the following indications or diseases comprising the steps of administration of an ozone therapeutic agent of the invention via insufflation, or treating red blood cells or whole blood ex vivo with one or more compositions of the present invention, followed by administering the prepared red blood cells or whole blood to said subject.

The most common types of anemia are iron deficiency anemia: Thalassaemia; Aplastic anemia; Hemolytic anemia; Sickle cell anemia; Pernicious anemia; Fanconi anemia

Sickle cell disease affects a considerable percentage of people that come from tropical or subtropical regions where malaria is or was common, or descendants thereof. It is a recessive autosomal genetic blood disorder, and presence of one sickle cell gene in a person enhances fitness against malaria. This has led to an evolutionary selection of people carrying one allele of the gene, in spite of the strong disadvantages for carriers of two alleles. Therefore, one third of the persons in Sub-Saharian Africa carry the gene.

Sickle cell disease affects, for example, approximately 100 000 individuals in the USA, mainly Afro-American children where one out of 500 born will have sickle cell anemia, a form of sickle cell disease (SCD). About 250 000 cases are born new world-wide.

In sickle cell disease, endothelial activation and leukocyte recruitment (caused by reperfusion injury, inflammatory stimuli, infection or hypersensitivity reactions) can lead to flow stasis, intravascular sickling of hemoglobin and vaso-occlusion, a hallmark of this disease. Inflammation is a recognized feature of SCD. Reperfusion injury (caused by vaso-occlusive events) and elevated levels of inflammatory cytokines in this disease are implicated in endothelial activation and inflammation. In transgenic sickle mice, exaggerated inflammatory response to hypoxia-reoxygenation is characterized by enhanced endothelial oxidant generation and leukocyte recruitment in venules (Kaul and Hebbel, J. Clin Invest. 106, 411-420, 2000). While during the hypoxic phase intravascular sickling is enhanced, reperfusion will induce exaggerated inflammatory response and leukocyte recruitment, which will promote flow stasis (Turhan et al. Proc. Natl. Acad. Sci. USA 99(5). 3047-3051, 2002).

Cancer, Tumors, Proliferative Diseases, Malignancies and their Metastases

The term “cancer” as used herein refers also to tumors, proliferative diseases, malignancies and their metastases. Examples for cancer diseases are adenocarcinoma, choroidal melanoma, acute leukemia, acoustic neurinoma, ampullary carcinoma, anal carcinoma, astrocytoma, basal cell carcinoma, pancreatic cancer, desmoid tumor, bladder cancer, bronchial carcinoma, non-small cell lung cancer (NSCLC), breast cancer, Burkitt's lymphoma, corpus cancer, CUP-syndrome (carcinoma of unknown primary), colorectal cancer, small intestine cancer, small intestinal tumors, ovarian cancer, endometrial carcinoma, ependymoma, epithelial cancer types, Ewing's tumors, gastrointestinal tumors, gastric cancer, gallbladder cancer, gall bladder carcinomas, uterine cancer, cervical cancer, cervix, glioblastomas, gynecologic tumors, ear, nose and throat tumors, hematologic neoplasias, hairy cell leukemia, urethral cancer, skin cancer, skin testis cancer, brain tumors (gliomas), brain metastases, testicle cancer, hypophysis tumor, carcinoids, Kaposi's sarcoma, laryngeal cancer, germ cell tumor, bone cancer, colorectal carcinoma, head and neck tumors (tumors of the ear, nose and throat area), colon carcinoma, craniopharyngiomas, oral cancer (cancer in the mouth area and on lips), cancer of the central nervous system, liver cancer, liver metastases, leukemia, eyelid tumor, lung cancer, lymph node cancer (Hodgkin's/Non-Hodgkin's), lymphomas, stomach cancer, malignant melanoma, malignant neoplasia, malignant tumors gastrointestinal tract, breast carcinoma, rectal cancer, medulloblastomas, melanoma, meningiomas, Hodgkin's disease, mycosis fungoides, nasal cancer, neurinoma, neuroblastoma, kidney cancer, renal cell carcinomas, non-Hodgkin's lymphomas, oligodendroglioma, esophageal carcinoma, osteolytic carcinomas and osteoplastic carcinomas, osteosarcomas, ovarial carcinoma, pancreatic carcinoma, penile cancer, plasmocytoma, squamous cell carcinoma of the head and neck (SCCHN), prostate cancer, pharyngeal cancer, rectal carcinoma, retinoblastoma, vaginal cancer, thyroid carcinoma, Schneeberger disease, esophageal cancer, spinalioms, T-cell lymphoma (mycosis fungoides), thymoma, tube carcinoma, eye tumors, urethral cancer, urologic tumors, urothelial carcinoma, vulva cancer, wart appearance, soft tissue tumors, soft tissue sarcoma, Wilm's tumor, cervical carcinoma and tongue cancer.

Infectious Disease

The immune system in higher vertebrates represents the first line of defense against various antigens that can enter the vertebrate body, including microorganisms such as bacteria, fungi and viruses that are the causative agents of a variety of diseases.

Despite large immunization programs, viral infections, such as influenza virus, human immunodeficiency virus (“HIV”), herpes simplex virus (“HSV”, type 1 or 2), human papilloma virus (“HPV”, type 16 or 18), human cytomegalovirus (“HCMV”) or human hepatitis B or C virus (“HBV”, Type B; “HCV”, type C) infections, remain a serious source of morbidity and mortality throughout the world and a significant cause of illness and death among people with immune-deficiency associated with aging or different clinical conditions. Although antiviral chemotherapy with compounds such as amantadine and rimantadine have been shown to reduce the duration of symptoms of clinical infections (i.e., influenza infection), major side effects and the emergence of drug-resistant variants have been described. New classes of antiviral agents designed to target particular viral proteins such as influenza neuraminidase are being developed. However, the ability of viruses to mutate the target proteins represents an obstacle for effective treatment with molecules which selectively inhibit the function of specific viral polypeptides. Thus, there is need for new therapeutic strategies to prevent and treat viral infections.

Additionally, there is a need for new therapies for the prevention and treatment of bacterial infections, especially bacterial infections caused by multiple drug resistant bacteria. Currently, bacterial infections are treated with various antibiotics. Although antibiotics have and can be effective in the treatment of various bacterial infections, there are a number of limitations to the effectiveness and safety of antibiotics. For example, some individuals have an allergic reaction to certain antibiotics and other individuals suffer from serious side effects. Moreover, continued use of antibiotics for the treatment of bacterial infections contributes to formation of antibiotic-resistant strains of bacteria.

Another aspect of the present invention is directed to the use of the therapeutic ozone agent for prophylaxis and/or treatment of infectious diseases including opportunistic infections.

Examples of infectious diseases are AIDS, alveolar hydatid disease (AHD, echinococcosis), amebiasis (Entamoeba histolytica infection), Angiostrongylus infection, anisakiasis, anthrax, babesiosis (Babesia infection), Balantidium infection (balantidiasis), Baylisascaris infection (raccoon roundworm), bilharzia (schistosomiasis), Blastocystis hominis infection (blastomycosis), boreliosis, botulism, Brainerd diarrhea, brucellosis, bovine spongiform encephalopathy (BSE), candidiasis, capillariasis (Capillaria infection), chronic fatigue syndrome (CFS), Chagas disease (American trypanosomiasis), chickenpox (Varicella-Zoster virus), Chlamydia pneumoniae infection, cholera, Creutzfeldt-Jakob disease (CJD), clonorchiasis (Clonorchis infection), cutaneous larva migrans (CLM) (hookworm infection), coccidioidomycosis, conjunctivitis, Coxsackievirus A16 (hand, foot and mouth disease), cryptococcosis, Cryptosporidium infection (cryptosporidiosis), Culex mosquito (West Nile virus vector), cyclosporiasis (Cyclospora infection), cysticercosis (neurocysticercosis), Cytomegalovirus infection, Dengue/Dengue fever, Dipylidium infection (dog and cat flea tapeworm), Ebola virus hemorrhagic fever, encephalitis, Entamoeba coli infection, Entamoeba dispar infection, Entamoeba hartmanni infection, Entamoeba histolytica infection (amebiasis), Entamoeba polecki infection, enterobiasis (pinworm infection), enterovirus infection (non-polio), Epstein-Barr virus infection, Escherichia coli infection, foodborne infection, foot and mouth disease, fungal dermatitis, gastroenteritis, group A streptococcal disease, group B streptococcal disease, Hansen's disease (leprosy), Hantavirus pulmonary syndrome, head lice infestation (pediculosis), Helicobacter pylori infection, hematologic disease, Hendra virus infection, hepatitis (HCV, HBV), herpes zoster (shingles), HIV Infection, human ehrlichiosis, human parainfluenza virus infection, influenza, isosporiasis (Isospora infection), Lassa fever, leishmaniasis, Kala-azar (Kala-azar, Leishmania Infection), lice (body lice, head lice, pubic lice), Lyme disease, malaria, Marburg hemorrhagic fever, measles, meningitis, mosquito-borne diseases, Mycobacterium avium complex (MAC) infection, Naegleria infection, nosocomial infections, nonpathogenic intestinal ameobae infection, onchocerciasis (river blindness), opisthorciasis (Opisthorcis infection), parvovirus infection, plague, Pneumocystis carinii pneumonia (PCP), polio, Q fever, rabies, respiratory syncytial virus (RSV) Infection, rheumatic fever, Rift Valley fever, river blindness (onchocerciasis), rotavirus infection, roundworm infection, salmonellosis, salmonella enteritidis, scabies, shigellosis, shingles, sleeping sickness, smallpox, streptococcal Infection, tapeworm infection (Taenia infection), tetanus, toxic shock syndrome, tuberculosis, ulcers (peptic ulcer disease), valley fever, Vibrio parahaemolyticus infection, Vibrio vulnificus infection, viral hemorrhagic fever, warts, waterborne infectious diseases, West Nile virus infection (West Nile encephalitis), whooping cough, yellow fever.

Another aspect of the present invention is directed to the use of the therapeutic ozone agent for prophylaxis and/or treatment of prion diseases.

Prions are infectious agents which do not have a nucleic acid genome. It seems that a protein alone is the infectious agent. A prion has been defined as “small proteinaceous infectious particle which resists inactivation by procedures that modify nucleic acids”. The discovery that proteins alone can transmit an infectious disease came as a considerable surprise to the scientific community. Prion diseases are often called “transmissible spongiform encephalopathies”, because of the post mortem appearance of the brain with large vacuoles in the cortex and cerebellum. Probably most mammalian species develop these diseases. Prion diseases are a group of neurodegenerative disorders of humans and animals and the prion diseases can manifest as sporadic, genetic or infectious disorders. Examples of prion diseases acquired by exogenous infection are bovine spongiform encephalitis (BSE) of cattle and the new variant of Creutzfeld-Jakob disease (vCJD) caused by BSE as well as scrapie of animals. Examples of human prion diseases include kuru, sporadic Creutzfeldt-Jakob disease (sCJD), familial CJD (fCJD), iatrogenic CJD (iCJD), Gerstmann-Straussler-Scheinker (GSS) disease, fatal familial insomnia (FFI), and especially the new variant CJD (nvCJD or vCJD).

The name “prion” is used to describe the causative agents which underlie the transmissible spongiform encephalopathies. A prion is proposed to be a novel infectious particle that differs from viruses and viroids. It is composed solely of one unique protein that resists most inactivation procedures such as heat, radiation, and proteases. The latter characteristic has led to the term protease-resistant isoform of the prion protein. The protease-resistant isoform has been proposed to slowly catalyze the conversion of the normal prion protein into the abnormal form.

The term “isoform” in the context of prions means two proteins with exactly the same amino acid sequence that can fold into molecules with dramatically different tertiary structures. The normal cellular isoform of the prion protein (PrP-C) has a high .alpha.-helix content, a low .beta.-sheet content, and is sensitive to protease digestion. The abnormal, disease-causing isoform (PrP-Sc) has a lower .alpha.-helix content, a much higher .beta.-sheet content, and is much more resistant to protease digestion.

As used herein the term “prion diseases” refers to transmissible spongiform encephalopathies. Examples for prion diseases comprise scrapie (sheep, goat), transmissible mink encephalopathy (TME; mink), chronic wasting disease (CWD; muledeer, deer, elk), bovine spongiform encephalopathy (BSE; cows, cattles), Creutzfeld-Jacob Disease (CJD), variant CJD (vCJD), sporadic Creutzfeldt-Jakob disease (sCJD), familial CJD (fCJD), iatrogenic CJD (iCJD, Gerstmann-Straussler-Scheinker syndrome (GSS), fatal familial insomnia (FFI), and kuru. Preferred are BSE, vCJD, and CJD.

Hepatitis B Virus (HBV) Infection

Hepatitis is an inflammation of the liver that is most often caused by infection with one of five viruses, hepatitis A, B, C, D or E. Hepatitis B virus infection may either be acute (self-limiting) or chronic (long-standing). Persons with self-limiting infection clear the infection spontaneously within weeks to months. In cases, particularly in those related to hepatitis B and C, “chronic hepatitis” may result. Chronic hepatitis occurs when the body is unable to completely clear the virus even though the symptoms may not persist.

Children are less likely than adults to clear the infection. More than 95% of people who become infected as adults or older children will stage a full recovery and develop protective immunity to the virus. However, only 5% of newborns that acquire the infection from their mother at birth will clear the infection. Of those infected between the age of one to six, 70% will clear the infection.

Acute infection with hepatitis B virus is associated with acute viral hepatitis—an illness that begins with general ill-health, loss of appetite, nausea, vomiting, body aches, mild fever, dark urine, and then progresses to development of jaundice. It has been noted that itchy skin has been an indication as a possible symptom of all hepatitis virus types. The illness lasts for a few weeks and then gradually improves in most affected people. A few patients may have more severe liver disease (fulminant hepatic failure), and may die as a result of it. The infection may be entirely asymptomatic and may go unrecognized.

Continued presence of the virus over a number of years can lead to cirrhosis (scarring of the liver). This type of infection dramatically increases the incidence of hepatocellular carcinoma (liver cancer). Chronic carriers should avoid consuming alcohol as it increases their risk for cirrhosis and liver cancer. It is estimated that approximately 350 million people worldwide are infected with chronic hepatitis B. HBV is transmitted through sexual contact, vertical transmission (mother to child at birth) or by coming into contact with contaminated blood. It is estimated that over 2 billion people worldwide have been infected with hepatitis B virus. Of these 2 billion, approximately 350 million people have developed chronic HBV infection, putting them at high risk of developing cirrhosis and liver cancer.

Hepatitis D infection can only occur with a concomitant infection with Hepatitis B virus because the Hepatitis D virus uses the Hepatitis B virus surface antigen to form a capsid. Co-infection with hepatitis D increases the risk of liver cirrhosis and liver cancer. Polyarteritis nodosa is more common in people with hepatitis B infection.

Chronic carriers of the HBV have been defined as those who are HBV surface antigen positive for greater than 6 months. Approximately 5-10% of those people who are infected with the virus will become carriers, an estimated 5-10% of those people infected each year will progress to chronic liver disease, cirrhosis and possibly liver cancer. About 5,000 people die in the United States each year related to HBV, 1,000 die of HBV-related liver cancer. The incubation period of HBV usually lasts from 2 to 4 months, although it may be very short (10 days) or extremely long (9 months).

The tests, called assays, for detection of hepatitis B virus infection involve serum or blood tests that detect either viral antigens (proteins produced by the virus) or antibodies produced by the host.

The hepatitis B surface antigen (HBsAg) is most frequently used to screen for the presence of this infection. It is the first detectable viral antigen to appear during infection. However, early in an infection, this antigen may not be present and it may be undetectable later in the infection as it is being cleared by the host. The infectious virion contains an inner “core particle” enclosing viral genome. The icoshedral core particle is made of 180 or 240 copies of core protein, alternatively known as hepatitis B core antigen, or HBcAg. During this ‘window’ in which the host remains infected but is successfully clearing the virus, IgM antibodies to the hepatitis B core antigen (anti-HBc IgM) may be the only serological evidence of disease.

Shortly after the appearance of the HBsAg, another antigen named as the hepatitis B e antigen (HBeAg) will appear. Traditionally, the presence of HBeAg in a host's serum is associated with much higher rates of viral replication and enhanced infectivity; however, variants of the hepatitis B virus do not produce the ‘e’ antigen, so this rule does not always hold true. During the natural course of an infection, the HBeAg may be cleared, and antibodies to the ‘e’ antigen (anti-HBe) will arise immediately afterwards. This conversion is usually associated with a dramatic decline in viral replication.

Individuals who remain HBsAg positive for at least six months are considered to be hepatitis B carriers. Carriers of the virus may have chronic hepatitis B, which would be reflected by elevated serum alanine aminotransferase levels and inflammation of the liver, as revealed by biopsy. Carriers who have seroconverted to HBeAg negative status, particularly those who acquired the infection as adults, have very little viral multiplication and hence may be at little risk of long-term complications or of transmitting infection to others. Therefore, the most significant event indicating a chronic course of hepatitis B is the absence of the HBsAg/anti-HBs seroconversion. If this phenomenon has not occurred within 6 months after the onset of the disease, persistence of the HBV infection and the related clinical pictures (asymptomatic HBsAg carrier, chronic hepatitis, cirrhosis, or hepatoma) have to be reckoned with.

More recently, PCR tests have been developed to detect and measure the amount of viral nucleic acid in clinical specimens. These tests are called viral loads and are used to assess a person's infection status and to monitor treatment.

Treatment of chronic infection is necessary to reduce the risk of cirrhosis and liver cancer. Chronically infected individuals with persistently elevated serum alanine aminotransferase, a marker of liver damage, and HBV DNA levels are candidates for therapy.

While monotherapy treatment with either Epivir-HBV (lamivudine, 3TC) or Intron A (interferon-alfa) shows some benefits for chronic hepatitis B, None of the available drugs can clear the infection, but they can help stop the virus from replicating, and prevent liver damage such as cirrhosis and liver cancer. Treatments include antiviral drugs such as lamivudine, adefovir and entecavir, telbivudine, and immune system modulators such as interferon alpha. However, some individuals are much more likely to respond than others and this might be because of the genotype of the infecting virus or the patient's heredity, and therefore there is a need for additional therapies. The virus is divided into four major serotypes (adr, adw, ayr, ayw) based on antigenic epitopes present on its envelope proteins, and into eight genotypes (A-H) according to overall nucleotide sequence variation of the genome. The genotypes have a distinct geographical distribution and are used in tracing the evolution and transmission of the virus. Differences between genotypes affect the disease severity, course and likelihood of complications, and response to treatment. The treatment works by reducing the viral load, (the amount of virus particles as measured in the blood), which in turn reduces viral replication in the liver.

Infants born to mothers known to carry hepatitis B can be treated with antibodies to the hepatitis B virus (hepatitis B immune globulin or HBIg). When given with the vaccine within twelve hours of birth, the risk of acquiring hepatitis B is reduced 95%. This treatment allows a mother to safely breastfeed her child.

The ozone and therapeutic ozone agent of the present invention were tested for their effect as active therapeutic agents in the prophylaxis and/or treatment of infectious diseases and disorders.

Autoimmune Disease

Autoimmune disease refers to any of a group of diseases or disorders in which tissue injury is associated with a humoral and/or cell-mediated immune response to body constituents or, in a broader sense, an immune response to self. The pathological immune response may be systemic or organ specific. That is, for example, the immune response directed to self may affect joints, skin, myelin sheath that protects neurons, kidney, liver, pancreas, thyroid, adrenals, and ovaries.

In fact, the list of autoimmune diseases is composed of more than eighty disorders. A few autoimmune diseases such as vitiligo, in which patches of skin lose pigmentation, are merely annoying. Most others are debilitating, often progressive with time and eventually fatal. Systemic lupus erythematosus (SLE), for example, is a chronic disease in which 10-15% of patients die within a decade of diagnosis, in all but a few autoimmune diseases, the sex ratio skews towards women. For example, in SLE the ratio of female to male patients is nine to one. In one particular case, Hashimoto's disease in which the immune system attacks the thyroid gland, the ratio is fifty to one.

It has long been known that immune complex formation plays a role in the etiology and progression of autoimmune disease. For example, inflammation in patients with arthritis has long been considered to involve phagocytosis by leukocytes of complexes of antigen, antibody and complement-immune complexes. However, only now it is being recognized that inflammation caused by immune complexes in the joints (arthritis), the kidneys (glomerulonephritis), and blood vessels (vasculitis) is a major cause of morbidity in autoimmune diseases. Increased immune complex formation correlates with the presence of antibodies directed to self or so-called autoantibodies, and the presence of the latter can also contribute to tissue inflammation either as part of an immune complex or unbound to antigen (free antibody). In some autoimmune diseases, the presence of free autoantibody contributes significantly to disease pathology. This has been clearly demonstrated for example in SLE (anti-DNA antibodies), immune thrombocytopenia (antibody response directed to platelets), and to a lesser extent rheumatoid arthritis (IgG reactive rheumatoid factor). The important role of immune complexes and free autoantibodies is further demonstrated by the fact that successful treatment of certain autoimmune diseases has been achieved by the removal of immune complexes and free antibody by means of specific immunoadsorption procedures. For example, the use of an apheresis procedure in which immune complexes and antibodies are removed by passage of a patient's blood through an immunoaffinity column was approved by the U.S. FDA in 1987 for immune thrombocytopenia (ITP) and in 1999 for rheumatoid arthritis. However, currently there is no approved method for the treatment of autoimmune diseases which facilitates the elimination of immune complexes and autoantibodies by administration of a drug.

Another aspect of the etiology and progression of autoimmune disease is the role of proinflammatory cytokines. Under normal circumstances, proinflammatory cytokines such as tumor necrosis factor .alpha. (TNF.alpha.) and interleukin-1 (IL-1) play a protective role in the response to infection and cellular stress. However, the pathological consequences which result from chronic and/or excessive production of TNF.alpha. and IL-1 are believed to underlie the progression of many autoimmune diseases such as rheumatoid arthritis, Crohn's disease, inflammatory bowel disease, and psoriasis. Other proinflammatory cytokines include interleukin-6, interleukin-8, interleukin-17, and granulocyte-macrophage colony stimulating factor.

Naturally occurring CD4+CD25+regulatory T cells (Tregs) play a critical role in the control of periphery tolerance to self-antigens. Interestingly, they also control immune responses to allergens and transplant antigens. Recent studies in animal models have shown that adoptive transfer of CD4+CD25+ Tregs can prevent or even cure allergic and autoimmune diseases, and appear to induce transplantation tolerance. Thus, adoptive cell therapy using patient-specific CD4+CD25+ Tregs has emerged as an individualized medicine for the treatment of inflammatory disease including allergy, autoimmune disease and transplant rejection. Furthermore, strategies to activate and expand antigen-specific CD4+CD25+ Tregs in vivo using pharmacological agents may represent a novel avenue for drug development.

The interaction of leukocytes with the vessel endothelium to facilitate the extravasation into the tissue represents a key process of the body's defense mechanisms. Excessive recruitment of leukocytes into the inflamed tissue in chronic diseases like autoimmune disorders could be prevented by interfering with the mechanisms of leukocyte extravasation. Significant progress in elucidating the molecular basis of the trafficking of leukocytes from the blood stream to the extravascular tissue has been achieved that enables new strategies for therapeutic approaches. The multistep process of leukocyte rolling, firm adhesion and transmigration through the endothelial wall is facilitated by a dynamic interplay of adhesion receptors on both leukocytes and on endothelial cells as well as chemokines. In preclinical studies using various animal models, promising results have been obtained demonstrating that blocking of adhesion receptors of the selectin and integrin families improved the inflammation process in models of ulcerative colitis, autoimmune encephalomyelitis or contact hypersensitivity. In addition to the targeting of adhesion receptors by antibodies, small molecules that mimic epitopes of adhesion receptor ligands have been developed and successfully applied in animal models. Clinical studies revealed a limited response using antibodies to selectins or leukocyte function-associated antigen 1 (LFA-1) integrins compared with animal models. However, using humanized antibodies to the alpha 4-integrin subunit significant efficacy has been demonstrated in autoimmune diseases like psoriasis, multiple sclerosis and inflammatory bowel disease.

Examples of autoimmune diseases of the eyes are idiopathic opticus-neuritis, ophthalmia sympathica, anterior uveitis and other uveitis forms, retina degeneration, and Mooren's ulcer.

Examples of autoimmune diseases of the skin are bullous pemphigoides, chronic urticaria (autoimmune subtype), dermatitis herpetiformis (morbus Duhring), epidermolysis bullosa aquisita (EBA), acquired angioedema, herpes gestationes, hypocomplementemic urticarial vasculitis syndrome (HUVS), linear IgA-dermatosis, and pemphigus.

Examples of hematological autoimmune diseases are autoimmune hemolytic anemia, autoimmune neutropenia, Evans syndrome, inhibitor hemophilia, idiopathic thrombocytopenial purpura (ITP) and pernicious anemia.

Examples of gynecological autoimmune diseases are habitual abortion and infertility.

Examples of autoimmune diseases of the heart are congenital heart block, idiopathic dilatative cardiomyopathy, peripartum-cardiomyopathy, postcardiotomy syndrome, and postinfarct syndrome (Dressler syndrome).

Examples of autoimmune diseases of the ear, nose and throat are chronic sensorineural hearing loss and morbus Meniere.

Examples of autoimmune diseases of the colon are autoimmune enteropathy, colitis ulcerosa, indeterminant colitis, Crohn's disease and gluten-sensitive enteropathy.

Examples of autoimmune endocrinological autoimmune disorders are autoimmune polyglandulary syndrome type 1, autoimmune polyglandulary syndrome type 2, diabetes mellitus type 1 (IDDM), Hashimoto-thyroiditis, insulin-autoimmune-syndrome (IAS), idiopathic diabetes insipidus, idiopathic hypoparathyroidism, idiopathic Addison's disease and Graves-Basedow disease.

Examples of autoimmune diseases of the liver are autoimmune hepatitis (AIH type 1, 2 and 3), primary biliary cirrhosis (PBC), and primary sclerosing cholangitis.

Example of autoimmune diseases of the lung is Goodpasture's syndrome. An example of an autoimmune disease of the stomach is chronic atrophic (type A) gastritis.

Examples of neurological autoimmune disorders are Guillain-Barre syndrome, IgM gammopathy-associated neuropathy, Lambert-Eaton syndrome, Miller-Fisher syndrome, multiple sclerosis, multifocal motoric neuropathy, myasthenia gravis, paraneoplastic neurological syndrome, Rasmussen's encephalitis, and stiff-man syndrome.

Examples of autoimmune diseases of the kidney are anti-TBM-nephritis, Goodpasture's syndrome/anti-GBM-nephritis, IgA-nephropathy, interstitial nephritis, and membrane proliferative glomerulonephritides.

Further diseases that may be caused by an autoimmune reaction are Behcet disease, chronic fatigue immune dysfunction syndrome (CFIDS), Cogan syndrome I, endometriosis, HELLP syndrome, Bechterew's disease, polymyalgia rheumatica, psoriasis, sarcoidosis and vitiligo.

During the last decade, new biotherapies have been developed for the treatment of systemic autoimmune diseases. The targets of these new treatments are all the steps of the immune response. These new therapies are: B lymphocyte (BL) inhibitors such as anti-CD20 monoclonal antibody, B lymphocyte stimulator (BLyS) antagonists and tolerogens of pathogenic-antibody secreting LB;

inhibitors of the costimulation between antigen-presenting cells and T lymphocyte (TL) like monoclonal anti-CD40 ligand antibody or CTLA4-Ig (abatecept); TL antagonists which can inhibit the proliferation of autoreactive T cells; cytokine antagonists; chemokine and adhesion antagonists which inhibit trafficking of immunocompetent cells to target organs. These new approaches are based on a better understanding of the autoimmune response.

The ozone and therapeutic ozone agent of the present invention were tested for their effect as active therapeutic agents in the prophylaxis and/or treatment of autoimmune diseases and disorders.

Fibrotic Disease

Fibrosis or fibrosis associated disorder affects the liver, epidermis, endodermis, muscle, tendon, cartilage, heart, pancreas, lung, uterus, nervous system, testis, ovary, adrenal gland, artery, vein, colon, small intestine, biliary tract, or stomach. In a further embodiment, the fibrosis or fibrosis associated disorder is interstitial lung fibrosis. In another embodiment the fibrosis or fibrosis associated disorder is the result of an infection with schistosoma. In another embodiment the fibrosis or fibrosis associated disorder is the result of wound healing.

Fibrosis is generally characterized by the pathologic or excessive accumulation of collagenous connective tissue. Fibrotic diseases and disorders include, but are not limited to, collagen disease, interstitial lung disease, human fibrotic lung disease (e.g., obliterative bronchiolitis, idiopathic pulmonary fibrosis, pulmonary fibrosis from a known etiology, tumor stroma in lung disease, systemic sclerosis affecting the lungs, Hermansky-Pudlak syndrome, coal worker's pneumoconiosis, asbestosis, silicosis, chronic pulmonary hypertension, AIDS associated pulmonary hypertension, sarcoidosis, and the like), fibrotic vascular disease, tubulointerstitial and glomerular fibrosis, myocardial fibrosis, arterial sclerosis, atherosclerosis, varicose veins, coronary infarcts, cerebral infarcts, myocardial fibrosis, musculoskeletal fibrosis, post-surgical adhesions, human kidney disease (e.g., nephritic syndrome, Alport's syndrome, HIV associated nephropathy, polycystic kidney disease, Fabry's disease, diabetic nephropathy, chronic glomerulonephritis, nephritis associated with systemic lupus, and the like), cutis keloid formation, progressive systemic sclerosis (PSS), primary sclerosing cholangitis (PSC), liver fibrosis, liver cirrhosis, renal fibrosis, pulmonary fibrosis, cystic fibrosis, chronic graft versus host disease, scleroderma (local and systemic), Grave's opthalmopathy, diabetic retinopathy, glaucoma, Peyronie's disease, penis fibrosis, urethrostenosis after a test using a cystoscope, inner accretion after surgery, scarring, myelofibrosis, idiopathic retroperitoneal fibrosis, peritoneal fibrosis from a known etiology, drug induced ergotism, fibrosis incident to benign or malignant cancer, fibrosis incident to microbial infection (e.g., viral, bacterial, parasitic, fungal, etc.), Alzheimer's disease, fibrosis incident to inflammatory bowel disease (including stricture formation in Crohn's disease and microscopic colitis), fibrosis induced by chemical or environmental insult (e.g., cancer chemotherapy, pesticides, radiation/cancer radiotherapy), and the like.

Diseases associated with fibrosis include lupus, graft versus host disease, scleroderma, systemic sclerosis, scleroderma-like disorders, sine scleroderma, calcinosis, Raynaud's esophageal dysfunction, sclerodactyly, telangiectasiae, hypersensitivity pneumonitis, collagen vascular disease, asthma, pulmonary arterial hypertension, glomerulonephritis, chronic obstructive pulmonary disease, fibrosis following myocardial infarction, central nervous system fibrosis following a stroke or neuro-degenerative diseases (e g Alzheimer's disease), proliferative vitreoretinopathy (PVR) and arthritis, silicosis, asbestos induced pulmonary fibrosis, acute lung injury and acute respiratory distress syndrome (including bacterial pneumonia induced, trauma induced, viral pneumonia induced, tuberculosis, ventilator induced, non-pulmonary sepsis induced, and aspiration induced). Increased Number of Activated Myofibroblasts in Fibrotic Diseases

The emergence and disappearance of the myofibroblast appears to correlate with the initiation of active fibrosis and its resolution, respectively. In addition, the myofibroblast has many phenotypic features, which embody much of the pathologic alterations in fibrotic tissue, e.g. lung tissue. These features would seem to argue for an important role for the myofibroblast in the pathogenesis of fibrosis, e.g. lung fibrosis. Furthermore, the persistence of the myofibroblast may herald progressive disease, and, conversely, its disappearance may be an indicator of resolution. This in turn suggests that future therapeutic strategies targeting the myofibroblast would be productive.

Patients usually exhibit evidence of active fibrosis with increased numbers of activated fibroblasts, many of which have the phenotypic characteristics of myofibroblasts. At these sites, increased amounts of extracellular matrix deposition are evident with effacement of the normal alveolar architecture. Animal model studies show the myofibroblast to be the primary source of type I collagen gene expression in active fibrotic sites. In vitro studies show differentiation of these cells from fibroblasts under the influence of certain cytokines but indicate their susceptibility to nitric oxide mediated apoptosis. In addition to promoting myofibroblast differentiation, transforming growth factor-β1 (TGF-β1) provides protection against apoptosis. Thus, this well-known fibrogenic cytokine is important both for the emergence of the myofibroblast and its survival against apoptotic stimuli. This is consistent with the critical importance of this cytokine in diverse models of fibrosis in various tissues. In view of these properties, the persistence or prolonged survival of the myofibroblast may be the key to understanding why certain forms of lung injury may result in progressive disease, terminating in end stage disease.

Although pulmonary fibrosis has diverse etiologies, there is a common feature characteristic of this process, namely, the abnormal deposition of extracellular matrix that effaces the normal lung tissue architecture. A key cellular source of this matrix is the mesenchymal cell population that occupies much of the fibrotic lesion during the active period of fibrosis. This population is heterogeneous with respect to a number of key phenotypes. One of these phenotypes is the myofibroblast, which is commonly identified by its expression in .alpha.-smooth muscle actin and by features that are intermediate between the bona fide smooth muscle cell and the fibroblast. The de novo appearance of myofibroblasts at sites of wound healing and tissue repair/fibrosis is associated with the period of active fibrosis and is considered to be involved in wound contraction. Furthermore, the localization of myofibroblasts at sites undergoing active extracellular matrix deposition suggests an important role for these cells in the genesis of the fibrotic lesion.

Increased TGF-β₁ Family Levels in Fibrotic Diseases

The transforming growth factor-131 (TGF-β₁) family of proteins has the most potent stimulatory effect on extracellular matrix deposition of any cytokines so far examined. In animal models of pulmonary fibrosis enhanced TGF-β1 gene expression is temporally and spatially related to increased collagen gene expression and protein deposition. TGF-β₁ antibodies reduce collagen deposition in murine bleomycin-induced lung fibrosis and human fibrotic lung tissue shows enhanced TGF-β₁ gene and protein expression. Several lines of evidence suggest that TGF-β is a central regulator of pulmonary fibrosis.

Diseases involving the lung associated with increased levels of TGF-β include chronic lung disease of prematurity, idiopathic pulmonary fibrosis, rapid progressive pulmonary fibrosis, giant-cell interstitial pneumonia, acute rejection after lung transplantation, cytomegalovirus pneumonitis after lung transplantation, bronchiolitis obliterans, asbestosis, coal worker's pneumoconiosis, silicosis, histiocytosis, sarcoidosis, eosinophilic granuloma, scleroderma, systemic lupus erythematosus, lymphangioleiomyomatosis, central fibrosis in pulmonary adenocarcinoma, cystic fibrosis, chronic obstructive lung disease, and asthma.

Increased TNF-α Levels in Fibrotic Diseases

An important role of tumor necrosis factor-α (TNF-α) in interstitial fibrosis has been established using transgenic mice, which either overexpress or display a deficiency of this cytokine. Mice transgenically modified to overexpress TNF-α develop lung fibrosis. In contrast, mice null for TNF-α show marked resistance to bleomycin induced fibrosis. TNF-α can stimulate fibroblast replication and collagen synthesis in vitro, and pulmonary TNF-α gene expression rises after administration of bleomycin in mice. Soluble TNF-α receptors reduce lung fibrosis in murine models and pulmonary overexpression of TNF-α in transgenic mice is characterized by lung fibrosis. In patients with CFA or asbestosis, bronchoalveolar lavage fluid-derived macrophages release increased amounts of TNF-α compared with controls.

Increased TNF-α may induce fibrosis or fibrosis-associated conditions affecting any tissue including, for example, fibrosis of an internal organ, a cutaneous or dermal fibrosing disorder, and fibrotic conditions of the eye. Fibrosis of internal organs (e.g., liver, lung, kidney, heart blood vessels, gastrointestinal tract) occurs in disorders such as pulmonary fibrosis, idiopathic fibrosis, autoimmune fibrosis, myelofibrosis, liver cirrhosis, veno-occlusive disease, mesangial proliferative glomerulonephritis, crescentic glomerulonephritis, diabetic nephropathy, renal interstitial fibrosis, renal fibrosis in subjects receiving cyclosporin, allograft rejection, HTV associated nephropathy. Other fibrosis-associated disorders include systemic sclerosis, eosinophilia-myalgia syndrome, and fibrosis-associated CNS disorders such as intraocular fibrosis. Dermal fibrosing disorders include, for example, scleroderma, morphea, keloids, hypertrophic scars, familial cutaneous collagenoma, and connective tissue nevi of the collagen type. Fibrotic conditions of the eye include conditions such as diabetic retinopathy, post-surgical scarring (for example, after glaucoma filtering surgery and after crossed-eyes (strabismus) surgery), and proliferative vitreoretinopathy. Additional fibrotic conditions that may be treated by the methods of the present invention may result, for example, from rheumatoid arthritis, diseases associated with prolonged joint pain and deteriorated joints; progressive systemic sclerosis, polymyositis, dermatomyositis, eosinophilic fascitis, morphea, Raynaud's syndrome, and nasal polyposis.

Increased Matrix Metalloproteases Levels in Fibrotic Diseases

The abnormal extracellular matrix (ECM) remodeling observed in the lungs of patients with interstitial pulmonary fibrosis (IPF) is due, at least in part, to an imbalance between matrix metalloproteases (MMPs) and tissue inhibitor of metalloproteinases (TIMPs). Normal lung fibroblasts do not make MMP-9 in vitro, whereas fibroblasts from IPF lungs strongly express MMP-9. In addition, fibroblasts from patients with IPF express increased levels of all TIMPs. In this setting, TIMPs may play a role in apoptosis in some cell populations. In vitro studies of alveolar macrophages obtained from untreated patients with idiopathic pulmonary fibrosis showed marked increase in MMP-9 secretion compared to macrophages collected from healthy individuals. In animals models of bleomycin-induced pulmonary fibrosis MMPs have been shown to be elevated in bronchoalveolar lavage (BAL) fluid. Indeed, a synthetic inhibitor of MMP, Batimastat, has been shown to significantly reduce bleomycin-induced lung fibrosis, again pointing to the importance of MMPs in the development of this fibrotic disease in the lung. A number of studies have shown that the actions of MMPs can result in the release of growth factors and cytokines. These profibrotic factors require proteolytic processing for their activation or release from extracellular matrix or carrier proteins before they can exert their activity. In fact, the proteolytic activity processing of several key factors involved in the pathogenesis of pulmonary fibrosis such as insulin-like growth factor (IGF), TGF-β1 and TNF-α occur through the actions of MMPs, thereby activating or releasing them from inhibitory protein-protein interactions. For example, IGFs in vivo are sequestered by six high affinity IGF binding proteins (IGFBPs1-6), preventing their ability to interact with IGF receptors. Studies examining adults and children IPF and interstitial lung disease show that beside IPF, IGFBP-3 and IFPB-2 levels are increased in IPF BAL fluid. MMPs have recently been shown to regulate the cleavage of IGF binding proteins, thereby liberating the complexed ligand to affect IGF actions in target cells. Observations have also shown that the gelatinases, MMP-9 and MMP-2 may be involved in proteolytic activation of latent TGF-.beta. complexes. Furthermore, the MMP inhibitor Batimastat reduces MMP-9 activity in BAL fluid, which was associated with decreased amount of TGF-β and TNF-α.

Pulmonary fibrosis can be an all too common consequence of an acute inflammatory response of the lung to a host of inciting events. Chronic lung injury due to fibrotic changes can result from an identifiable inflammatory event or an insidious, unknown event. The inflammatory process can include infiltration of various inflammatory cell types, such as neutrophils and macrophages, the secretion of inflammatory cytokines and chemokines and the secretion of matrix remodeling proteinases.

Increased CCL18 Levels in Fibrotic Diseases

The expression and regulation of cysteine-cysteine (CC) chemokine ligand 18 (CCL18), a marker of alternative activation, by human alveolar macrophages (AMs) is increased in patients with pulmonary fibrosis and correlates negatively with pulmonary function test parameters. Thus, CCL18 is an ideal diagnostic marker for pulmonary fibrosis.

### Inflammatory Disease

Inflammation is the final common pathway of various insults, such as infection, trauma, and allergies to the human body. It is characterized by the activation of the immune system with recruitment of inflammatory cells, production of pro-inflammatory cells and production of pro-inflammatory cytokines. Most inflammatory diseases and disorders are characterized by abnormal accumulation of inflammatory cells including monocytes/macrophages, granulocytes, plasma cells, lymphocytes and platelets. Along with tissue endothelial cells and fibroblasts, these inflammatory cells release a complex array of lipids, growth factors, cytokines and destructive enzymes that cause local tissue damage.

One form of inflammatory response is neutrophilic inflammation which is characterized by infiltration of the inflamed tissue by neutrophil polymorphonuclear leukocytes (PMN), which are a major component of the host defense. Tissue infection by extracellular bacteria represents the prototype of this inflammatory response. On the other hand, various non-infectious diseases are characterized by extravascular recruitment of neutrophils. This group of inflammatory diseases includes chronic obstructive pulmonary disease, adult respiratory distress syndrome, some types of immune-complex alveolitis, cystic fibrosis, bronchitis, bronchiectasis, emphysema, glomerulonephritis, rheumatoid arthritis, gouty arthritis, ulcerative colitis, certain dermatoses such as psoriasis and vasculitis. In these conditions neutrophils are thought to play a crucial role in the development of tissue injury which, when persistent, can lead to the irreversible destruction of the normal tissue architecture with consequent organ dysfunction. Tissue damage is primarily caused by the activation of neutrophils followed by their release of proteinases and increased production of oxygen species.

Chronic obstructive pulmonary disease (COPD) is described by the progressive development of airflow limitation that is not fully reversible. Most patients with COPD have three pathological conditions; bronchitis, emphysema and mucus plugging. This disease is characterized by a slowly progressive and irreversible decrease in forced expiratory volume in the first second of expiration (FEVi), with relative preservation of forced vital capacity (FVC). In both asthma and COPD there is significant, but distinct, remodeling of airways. Most of the airflow obstruction is due to two major components, alveolar destruction (emphysema) and small airways obstruction (chronic obstructive bronchitis). COPD is mainly characterized by profound mucus cell hyperplasia. Neutrophil infiltration of the patient's lungs is a primary characteristic of COPD. Elevated levels of proinflammatory cytokines, like TNF-.alpha., and especially chemokines like interleukin-8 (IL-8) and growth-regulated oncogene-.alpha. (GRO-.alpha.) play a very important role in pathogenesis of this disease. Platelet thromboxane synthesis is also enhanced in patients with COPD. Most of the tissue damage is caused by activation of neutrophils followed by their release of metalloproteinases, and increased production of oxygen species.

TNF-α has several biologic activities that are important in homeostasis as well as in pathophysiological conditions. The main sources of TNF-α are monocytes-macrophages, T-lymphocytes and mast cells. The finding that anti-TNF-α antibodies (cA2) are effective in the treatment of patients suffering from rheumatoid arthritis (RA) intensified the interest to find new TNF-α. inhibitors as possible potent medicaments for RA. Rheumatoid arthritis is an autoimmune chronic inflammatory disease characterized by irreversible pathological changes of the joints. In addition to RA, TNF-α antagonists are also applicable to several other pathological conditions and diseases such as spondylitis, osteoarthritis, gout and other arthritic conditions, sepsis, septic shock, toxic shock syndrome, atopic dermatitis, contact dermatitis, psoriasis, glomerulonephritis, lupus erythematosus, scleroderma, asthma, cachexia, chronic obstructive lung disease, congestive heart failure, insulin resistance, lung (pulmonary) fibrosis, multiple sclerosis, Crohn's disease, ulcerative colitis, viral infections and AIDS.

The term “immunoinflammatory disorder” encompasses a variety of conditions, including autoimmune diseases, proliferative skin diseases, and inflammatory dermatoses. Immunoinflammatory disorders result in the destruction of healthy tissue by an inflammatory process, dysregulation of the immune system, and unwanted proliferation of cells. Examples of immunoinflammatory disorders are acne vulgaris; acute respiratory distress syndrome; Addison's disease; allergic rhinitis; allergic intraocular inflammatory diseases, antineutrophil cytoplasmic antibody (ANCA)-associated small-vessel vasculitis; ankylosing spondylitis; arthritis, asthma; atherosclerosis; atopic dermatitis; autoimmune hepatitis; autoimmune hemolytic anemia; autoimmune hepatitis; Behcet's disease; Bell's palsy; bullous pemphigoid; cerebral ischemia; chronic obstructive pulmonary disease; cirrhosis; Cogan's syndrome; contact dermatitis; COPD; Crohn's disease; Cushing's syndrome; dermatomyositis; diabetes mellitus; discoid lupus erythematosus; eosinophilic fasciitis; erythema nodosum; exfoliative dermatitis; fibromyalgia; focal glomerulosclerosis; focal segmental glomerulosclerosis; giant cell arteritis; gout; gouty arthritis; graft versus host disease; hand eczema; Henoch-Schonlein purpura; herpes gestationis; hirsutism; idiopathic cerato-scleritis; idiopathic pulmonary fibrosis; idiopathic thrombocytopenic purpura; immune thrombocytopenic purpura inflammatory bowel or gastrointestinal disorders, inflammatory dermatoses; lichen planus; lupus nephritis; lymphomatous tracheobronchitis; macular edema; multiple sclerosis; myasthenia gravis; myositis; nonspecific fibrosing lung disease; osteoarthritis; pancreatitis; pemphigoid gestationis; pemphigus vulgaris; periodontitis; polyarteritis nodosa; polymyalgia rheumatica; pruritus scroti; pruritis/inflammation, psoriasis; psoriatic arthritis; pulmonary histoplasmosis; rheumatoid arthritis; relapsing polychondritis; rosacea caused by sarcoidosis; rosacea caused by scleroderma; rosacea caused by Sweet's syndrome; rosacea caused by systemic lupus erythematosus; rosacea caused by urticaria; rosacea caused by zoster-associated pain; sarcoidosis; scleroderma; segmental glomerulosclerosis; septic shock syndrome; shoulder tendinitis or bursitis; Sjogren's syndrome; Still's disease; stroke-induced brain cell death; Sweet's disease; systemic lupus erythematosus; systemic sclerosis; Takayasu's arteritis; temporal arteritis; toxic epidermal necrolysis; transplant-rejection and transplant-rejection-related syndromes; tuberculosis; type-1 diabetes; ulcerative colitis; uveitis; vasculitis; and Wegener's granulomatosis.

As used herein, “non-dermal inflammatory disorders” include, for example, rheumatoid arthritis, inflammatory bowel disease, asthma, and chronic obstructive pulmonary disease. By “dermal inflammatory disorders” or “inflammatory dermatoses” is meant an inflammatory disorder selected from psoriasis, guttate psoriasis, inverse psoriasis, pustular psoriasis, erythrodermic psoriasis, acute febrile neutrophilic dermatosis, eczema, asteatotic eczema, dyshidrotic eczema, vesicular palmoplantar eczema, acne vulgaris, atopic dermatitis, contact dermatitis, allergic contact dermatitis, dermatomyositis, exfoliative dermatitis, hand eczema, pompholyx, rosacea, rosacea caused by sarcoidosis, rosacea caused by scleroderma, rosacea caused by Sweet's syndrome, rosacea caused by systemic lupus erythematosus, rosacea caused by urticaria, rosacea caused by zoster-associated pain, Sweet's disease, neutrophilic hidradenitis, sterile pustulosis, drug eruptions, seborrheic dermatitis, pityriasis rosea, cutaneous kikuchi disease, pruritic urticarial papules and plaques of pregnancy, Stevens-Johnson syndrome and toxic epidermal necrolysis, tattoo reactions, Wells syndrome (eosinophilic cellulitis), reactive arthritis (Reiter's syndrome), bowel-associated dermatosis-arthritis syndrome, rheumatoid neutrophilic dermatosis, neutrophilic eccrine hidradenitis, neutrophilic dermatosis of the dorsal hands, balanitis circumscripta plasmacellularis, balanoposthitis, Behcet's disease, erythema annulare centrifugum, erythema dyschromicum perstans, erythema multiforme, granuloma annulare, hand dermatitis, lichen nitidus, lichen planus, lichen sclerosus et atrophicus, lichen simplex chronicus, lichen spinulosus, nummular dermatitis, pyoderma gangrenosum, sarcoidosis, subcorneal pustular dermatosis, urticaria, and transient acantholytic dermatosis.

By “proliferative skin disease” is meant a benign or malignant disease that is characterized by accelerated cell division in the epidermis or dermis. Examples of proliferative skin diseases are psoriasis, atopic dermatitis, nonspecific dermatitis, primary irritant contact dermatitis, allergic contact dermatitis, basal and squamous cell carcinomas of the skin, lamellar ichthyosis, epidermolytic hyperkeratosis, premalignant keratosis, acne, and seborrheic dermatitis. As will be appreciated by one skilled in the art, a particular disease, disorder, or condition may be characterized as being both a proliferative skin disease and an inflammatory dermatosis. An example of such a disease is psoriasis.

Symptoms and signs of inflammation associated with specific conditions include: rheumatoid arthritis:—pain, swelling, warmth and tenderness of the involved joints; generalized and morning stiffness; insulin-dependent diabetes mellitus-insulitis; this condition can lead to a variety of complications with an inflammatory component, including:—retinopathy, neuropathy, nephropathy; coronary artery disease, peripheral vascular disease, and cerebrovascular disease; autoimmune thyroiditis:—weakness, constipation, shortness of breath, puffiness of the face, hands and feet, peripheral edema, bradycardia; multiple sclerosis:—spasticity, blurry vision, vertigo, limb weakness, paresthesias; uveoretinitis:—decreased night vision, loss of peripheral vision; lupus erythematosus:—joint pain, rash, photosensitivity, fever, muscle pain, puffiness of the hands and feet, abnormal urinalysis (hematuria, cylinduria, proteinuria), glomerulonephritis, cognitive dysfunction, vessel thrombosis, pericarditis; scleroderma:—Raynaud's disease; swelling of the hands, arms, legs and face; skin thickening; pain, swelling and stiffness of the fingers and knees, gastrointestinal dysfunction, restrictive lung disease; pericarditis; renal failure; other arthritic conditions having an inflammatory component such as rheumatoid spondylitis, osteoarthritis, septic arthritis and polyarthritis:—fever, pain, swelling, tenderness; other inflammatory brain disorders, such as meningitis, Alzheimer's disease, AIDS dementia encephalitis:—photophobia, cognitive dysfunction, memory loss; other inflammatory eye inflammations, such as retinitis:—decreased visual acuity; inflammatory skin disorders, such as, eczema, other dermatitis (e.g., atopic, contact), psoriasis, burns induced by UV radiation (sun rays and similar UV sources):—erythema, pain, scaling, swelling, tenderness; inflammatory bowel disease, such as Crohn's disease, ulcerative colitis:—pain, diarrhea, constipation, rectal bleeding, fever, arthritis; asthma:—shortness of breath, wheezing; other allergy disorders, such as allergic rhinitis:—sneezing, itching, runny nose conditions associated with acute trauma such as cerebral injury following stroke-sensory loss, motor loss, cognitive loss; heart tissue injury due to myocardial ischemia:—pain, shortness of breath; lung injury such as that which occurs in adult respiratory distress syndrome:—shortness of breath, hyperventilation, decreased oxygenation, pulmonary infiltrates; inflammation accompanying infection, such as sepsis, septic shock, toxic shock syndrome:—fever, respiratory failure, tachycardia, hypotension, leukocytosis; other inflammatory conditions associated with particular organs or tissues, such as: (i) nephritis (e.g., glomeralonephritis): —oliguria, abnormal urinalysis; (ii) inflamed appendix:—fever, pain, tenderness, leukocytosis; (iii) gout:—pain, tenderness, swelling and erythema of the involved joint, elevated serum and/or urinary uric acid; (iv) inflamed gall bladder:—abdominal pain and tenderness, fever, nausea, leukocytosis; (v) congestive heart failure:—shortness of breath, rales, peripheral edema; (vi) Type II diabetes:—end organ complications including cardiovascular, ocular, renal, and peripheral vascular disease; (vii) lung (pulmonary) fibrosis:—hyperventilation, shortness of breath, decreased oxygenation; (viii) vascular disease, such as atherosclerosis and restenosis: —pain, loss of sensation, diminished pulses, loss of function; and (ix) alloimmunity leading to transplant rejection:—pain, tenderness, fever.

Neurodegenerative Disease

The present invention also relates generally to the fields of neurology and psychiatry and to methods of protecting and/or the cells of a mammalian central nervous system from damage or injury.

Injuries or trauma of various kinds to the central nervous system (CNS) or the peripheral nervous system (PNS) can produce profound and long-lasting neurological and/or psychiatric symptoms and disorders. One form that this can take is the progressive death of neurons or other cells of the central nervous system (CNS), i.e., neurodegeneration or neuronal degeneration.

Neuronal degeneration as a result of, for example; Alzheimer's disease, multiple sclerosis, cerebral-vascular accidents (CVAs)/stroke, traumatic brain injury, spinal cord injuries, degeneration of the optic nerve, e.g., ischemic optic neuropathy or retinal degeneration and other central nervous system disorders is an enormous medical and public health problem by virtue of both its high incidence and the frequency of long-term sequalae. Animal studies and clinical trials have shown that amino acid transmitters (especially glutamate), oxidative stress and inflammatory reactions contribute strongly to cell death in these conditions. Upon injury or upon ischemic insult, damaged neurons release massive amounts of the neurotransmitter glutamate, which is excitotoxic to the surrounding neurons. Glutamate is a negatively charged amino acid that is an excitatory synaptic transmitter in the mammalian nervous system. Although the concentration of glutamate can reach the millimolar range in nerve terminals its extracellular concentration is maintained at a low level to prevent neurotoxicity. It has been noted that glutamate can be toxic to neurons if presented at a high concentration. The term “excitotoxicity” has been used to describe the cytotoxic effect that glutamate (and other such excitatory amino acids) can have on neurons when applied at high dosages.

Patients with injury or damage of any kind to the central (CNS) or peripheral (PNS) nervous system including the retina may benefit from neuroprotective methods of theinvention. This nervous system injury may take the form of an abrupt insult or an acute injury to the nervous system as in, for example, acute neurodegenerative disorders including, but not limited to; acute injury, hypoxia-ischemia or the combination thereof resulting in neuronal cell death or compromise. Acute injury includes, but is not limited to, traumatic brain injury (TBI) including, closed, blunt or penetrating brain trauma, focal brain trauma, diffuse brain damage, spinal cord injury, intracranial or intravertebral lesions (including, but not limited to, contusion, penetration, shear, compression or laceration lesions of the spinal cord or whiplash shaken infant syndrome).

In addition, deprivation of oxygen or blood supply in general can cause acute injury as in hypoxia and/or ischemia including, but not limited to, cerebrovascular insufficiency, cerebral ischemia or cerebral infarction (including cerebral ischemia or infarctions originating from embolic occlusion and thrombosis, retinal ischemia (diabetic or otherwise), glaucoma, retinal degeneration, multiple sclerosis, toxic and ischemic optic neuropathy, reperfusion following acute ischemia, perinatal hypoxic-ischemic injury, other hypoxic conditions, cardiac arrest or intracranial hemorrhage of any type (including, but not limited to, epidural, subdural, subarachnoid or intracerebral hemorrhage).

Trauma or injury to tissues of the nervous system may also take the form of more chronic and progressive neurodegenerative disorders, such as those associated with progressive neuronal cell death or compromise over a period of time including, but not limited to, Alzheimer's disease, Picks disease, diffuse Lewy body disease, progressive supranuclear palsy (Steel-Richardson syndrome), multisystem degeneration (Shy-Drager syndrome), chronic epileptic conditions associated with neurodegeneration, motor neuron diseases (amyotrophic lateral sclerosis), multiple sclerosis, degenerative ataxias, cortical basal degeneration, ALS-Parkinson's-dementia complex of Guam, subacute sclerosing panencephalitis, Huntington's disease, Parkinson's disease, synucleinopathies (including multiple system atrophy), primary progressive aphasia, striatonigral degeneration, Machado-Joseph disease or spinocerebellar ataxia type 3 and olivopontocerebellar degenerations, bulbar and pseudobulbar palsy, spinal and spinobulbar muscular atrophy (Kennedy's disease), primary lateral sclerosis, familial spastic paraplegia, Werdnig-Hoffmann disease, Kugelberg-Welander disease, Tay-Sach's disease, Sandhoff disease, familial spastic disease, Wohlfart-Kugelberg-Welander disease, spastic paraparesis, progressive multifocal leukoencephalopathy, familial dysautonomia (Riley-Day syndrome) or prion diseases (including, but not limited to Creutzfeld-Jakob disease, Gerstmann-Strussler-Scheinker disease, Kuru disease or fatal familial insomnia).

In addition, trauma and progressive injury to the nervous system can take place in various psychiatric disorders, including but not limited to, progressive, deteriorating forms of bipolar disorder or schizoaffective disorder or schizophrenia, impulse control disorders, obsessive compulsive disorder (OCD), behavioral changes in temporal lobe epilepsy and personality disorders.

In one preferred embodiment the compounds of the invention would be used to provide neuroprotection in disorders involving trauma and progressive injury to the nervous system in various psychiatric disorders. These disorders would be selected from the group consisting of; schizoaffective disorder, schizophrenia, impulse control disorders, obsessive compulsive disorder (OCD) and personality disorders.

In addition, trauma and injury make take the form of disorders associated with overt and extensive memory loss including, but not limited to, neurodegenerative disorders associated with age-related dementia, vascular dementia, diffuse white matter disease (Binswanger's disease), dementia of endocrine or metabolic origin, dementia of head trauma and diffuse brain damage, dementia Pugilistica or frontal lobe dementia, including but not limited to Picks Disease.

The invention further includes a method of treating Alzheimer's disease in a patient, preferably a human, comprising administering to a patient a therapeutic ozone agent capable of, for example, ablating or alleviating inflammatory processes in the brain associated with AD, which are diminished, thereby ablating or alleviating AD.

As used herein, AD is “alleviated” if the severity of a symptom of the AD, the frequency with which such a symptom is experienced by a patient, or both, are reduced. As used herein, AD is “ablated” if the symptoms of AD have largely disappeared in the patient.

There is also included in the invention a method of preventing AD in patient comprising administering to a patient known to be at risk for AD a therapeutic ozone agent capable of capable of, for example, ablating or alleviating inflammatory processes in the brain associated with AD, which are diminished, thereby preventing AD in a patient.

The Alzheimer's disease is optionally mild Alzheimer's disease, moderate Alzheimer's disease or severe Alzheimer's disease.

Overproduction and accumulation of amyloid beta is a pathologic feature of Alzheimer's disease. Human amyloid beta (Abeta) is the main component of insoluble amyloid plaques-deposits found in the brain of patients with Alzheimer's disease. The plaques are composed of fibrillar aggregates of Abeta. Amyloid beta fibrils have been associated with the advanced stages of Alzheimer's disease.

The cognitive hallmark of early Alzheimer's disease (AD) is an extraordinary inability to form new memories. Early memory loss is considered a synapse failure caused by soluble AP oligomers. These oligomers block long-term potentiation, a classic experimental paradigm for synaptic plasticity, and they are strikingly elevated in AD brain tissue and transgenic AD models. It has been hypothesized that early memory loss stems from synapse failure before neuron death and that synapse failure derives from actions of soluble Aβ oligomers rather than fibrils. Lacor et al., Synaptic targeting by Alzheimer's-related amyloid oligomers, J. Neurosci. 2004, 24(45):10191-10200.

Other disorders associated with neuronal injury include, but are not limited to, disorders associated with chemical, toxic, infectious and radiation injury of the nervous system including the retina, injury during fetal development, prematurity at time of birth, anoxic-ischemia, injury from hepatic, glycemic, uremic, electrolyte and endocrine origin, injury of psychiatric origin (including, but not limited to, psychopathology, depression or anxiety), injury from peripheral diseases and plexopathies (including plexus palsies) or injury from neuropathy (including neuropathy selected from multifocal, sensory, motor, sensory-motor, autonomic, sensory-autonomic or demyelinating neuropathies (including, but not limited to Guillain-Barre syndrome or chronic inflammatory demyelinating polyradiculoneuropathy) or those neuropathies originating from infections, inflammation, immune disorders, drug abuse, pharmacological treatments, toxins, trauma (including, but not limited to compression, crush, laceration or segmentation traumas), metabolic disorders (including, but not limited to, endocrine or paraneoplastic), Charcot-Marie-Tooth disease (including, but not limited to, type 1a, 1b, 2, 4a or 1-X linked), Friedreich's ataxia, metachromatic leukodystrophy, Refsum's disease, adrenomyeloneuropathy, ataxia-telangiectasia, Djerine-Sottas (including, but not limited to, types A or B), Lambert-Eaton syndrome or disorders of the cranial nerves).

Further indications are cognitive disorders. The term “cognitive disorder” shall refer to anxiety disorders, delirium, dementia, amnestic disorders, dissociative disorders, eating disorders, mood disorders, schizophrenia, psychotic disorders, sexual and gender identity disorders, sleep disorders, somatoform disorders, acute stress disorder, obsessive-compulsive disorder, panic disorder, posttraumatic stress disorder, specific phobia, social phobia, substance withdrawal delirium, Alzheimer's disease, Creutzfeldt-Jakob disease, head trauma, Huntington's disease, HIV disease, Parkinson's disease, Pick's disease, learning disorders, motor skills disorders, developmental coordination disorder, communication disorders, phonological disorder, pervasive developmental disorders, Asperger's disorder, autistic disorder, childhood disintegrative disorder, Rett's disorder, pervasive developmental disorder, attention-deficit/hyperactivity disorder (ADHD), conduct disorder, oppositional defiant disorder, pica, rumination disorder, tic disorders, chronic motor or vocal tic disorder, Tourette's disorder, elimination disorders, encopresis, enuresis, selective mutism, separation anxiety disorder, dissociative amnesia, depersonalization disorder, dissociative fugue, dissociative identity disorder, anorexia nervosa, bulimia nervosa, bipolar disorders, schizophreniform disorder, schizoaffective disorder, delusional disorder, psychotic disorder, shared psychotic disorder, delusions, hallucinations, substance-induced psychotic disorder, orgasmic disorders, sexual pain disorders, dyspareunia, vaginismus, sexual dysfunction, paraphilias, dyssomnias, breathing-related sleep disorder, circadian rhythm sleep disorder, hypersomnia, insomnia, narcolepsy, dyssomnia, parasomnias, nightmare disorder, sleep tenor disorder, sleepwalking disorder, parasomnia, body dysmorphic disorder, conversion disorder, hypochondriasis, pain disorder, somatization disorder, alcohol related disorders, amphetamine related disorders, caffeine related disorders, cannabis related disorders, cocaine related disorders, hallucinogen related disorders, inhalant related disorders, nicotine related disorders, opioid related disorders, phencyclidine-related disorder, abuse, persisting amnestic disorder, intoxication, withdrawal.

The term “bipolar and clinical disorders” shall refer to adjustment disorders, anxiety disorders, delirium, dementia, amnestic and other cognitive disorders, disorders usually first diagnosed in infancy (e.g.), childhood, or adolescence, dissociative disorders (e.g. dissociative amnesia, depersonalization disorder, dissociative fugue and dissociative identity disorder), eating disorders, factitious disorders, impulse-control disorders, mental disorders due to a general medical condition, mood disorders, other conditions that may be a focus of clinical attention, personality disorders, schizophrenia and other psychotic disorders, sexual and gender identity disorders, sleep disorders, somatoform disorders, substance-related disorders, generalized anxiety disorder (e.g. acute stress disorder, posttraumatic stress disorder), panic disorder, phobia, agoraphobia, obsessive-compulsive disorder, stress, acute stress disorder, anxiety neurosis, nervousness, phobia, posttraumatic stress disorder, posttraumatic stress disorder (PTSD), abuse, obsessive-compulsive disorder (OCD), manic depressive psychosis, specific phobias, social phobia, adjustment disorder with anxious features.

Examples for disorders usually first diagnosed in infancy, childhood, or adolescence are: mental retardation, learning disorders, mathematics disorder, reading disorder, disorder of written expression, motor skills disorders, developmental coordination disorder, communication disorders, expressive language disorder, phonological disorder, mixed receptive-expressive language disorder, stuttering, pervasive developmental disorders, Asperger's disorder, autistic disorder, childhood disintegrative disorder, Rett's disorder, pervasive developmental disorder, attention-deficit/hyperactivity disorder (ADHD), conduct disorder, oppositional defiant disorder, feeding disorder of infancy or early childhood, pica, rumination disorder, tic disorders, chronic motor or vocal tic disorder, Tourette's syndrome, elimination disorders, encopresis, enuresis, selective mutism, separation anxiety disorder, reactive attachment disorder of infancy or early childhood, stereotypic movement disorder.

Examples for substance-related disorders are: alcohol related disorders, amphetamine related disorders, caffeine related disorders, cannabis related disorders, cocaine related disorders, hallucinogen related disorders, inhalant related disorders, nicotine related disorders, opioid related disorders, psychotic disorder, psychotic disorder, phencyclidine-related disorder, abuse, persisting amnestic disorder, anxiety disorder, persisting dementia, dependence, intoxication, intoxication delirium, mood disorder, psychotic disorder, withdrawal, withdrawal delirium, sexual dysfunction, sleep disorder.

The term “neuroprotection” as used herein shall mean; inhibiting, preventing, ameliorating or reducing the severity of the dysfunction, degeneration or death of nerve cells, axons or their supporting cells in the central or peripheral nervous system of a mammal, including a human. This includes the treatment or prophylaxis of a neurodegenerative disease; protection against excitotoxicity or ameliorating the cytotoxic effect of a compound (for example, a excitatory amino acid such as glutamate; a toxin; or a prophylactic or therapeutic compound that exerts an immediate or delayed cytotoxic side effect including but not limited to the immediate or delayed induction of apoptosis) in a patient in need thereof.

The term “a patient in need of treatment with a neuroprotective agent” as used herein will refer to any patient who currently has or may develop any of the above syndromes or disorders, or any disorder in which the patient's present clinical condition or prognosis could benefit from providing neuroprotection to prevent the development, extension, worsening or increased resistance to treatment of any neurological or psychiatric disorder.

The term “treating” or “treatment” as used herein, refers to any indicia of success in the prevention or amelioration of an injury, pathology or condition, including any objective or subjective parameter such as abatement; remission; diminishing of symptoms or making the injury, pathology, or condition more tolerable to the patient; slowing in the rate of degeneration or decline; making the final point of degeneration less debilitating; or improving a subject's physical or mental well-being. The treatment or amelioration of symptoms can be based on objective or subjective parameters; including the results of a physical examination, neurological examination, and/or psychiatric evaluations.

In some embodiments this invention provides methods of neuroprotection. In certain embodiments, these methods comprise administering a therapeutically effective amount of the therapeutic ozone agent of the invention to a patient who has not yet developed overt, clinical signs or symptoms of injury or damage to the cells of the nervous system but who may be in a high risk group for the development of neuronal damage because of injury or trauma to the nervous system or because of some known predisposition either biochemical or genetic or the finding of a verified biomarker of one or more of these disorders.

Thus, in some embodiments, the methods and compositions of the present invention are directed toward neuroprotection in a subject who is at risk of developing neuronal damage but who has not yet developed clinical evidence. This patient may simply be at “greater risk” as determined by the recognition of any factor in a subject's, or their families, medical history, physical exam or testing that is indicative of a greater than average risk for developing neuronal damage. Therefore, this determination that a patient may be at a “greater risk” by any available means can be used to determine whether the patient should be treated with the methods of the present invention.

Accordingly, in an exemplary embodiment, subjects who may benefit from treatment by the methods and ozone agent of this invention can be identified using accepted screening methods to determine risk factors for neuronal damage. These screening methods include, for example, conventional work-ups to determine risk factors including but not limited to: for example, head trauma, either closed or penetrating, CNS infections, bacterial or viral, cerebrovascular disease including but not limited to stroke, brain tumors, brain edema, cysticercosis, porphyria, metabolic encephalopathy, drug withdrawal including but not limited to sedative-hypnotic or alcohol withdrawal, abnormal perinatal history including anoxia at birth or birth injury of any kind, cerebral palsy, learning disabilities, hyperactivity, history of febrile convulsions as a child, history of status epilepticus, family history of epilepsy or any seizure related disorder, inflammatory disease of the brain including lupis, drug intoxication either direct or by placental transfer, including but not limited to cocaine poisoning, parental consanguinity, and treatment with medications that are toxic to the nervous system including psychotropic medications.

As used herein, the terms “surrogate marker” and “biomarker” are used interchangeably and refer to any anatomical, biochemical, structural, electrical, genetic or chemical indicator or marker that can be reliably correlated with the present existence or future development of neuronal damage. In some instances, brain-imaging techniques, such as computer tomography (CT), magnetic resonance imaging (MRI) or positron emission tomography (PET), can be used to determine whether a subject is at risk for neuronal damage. Suitable biomarkers for the methods of this invention include, but are not limited to: the determination by MRI, CT or other imaging techniques, of sclerosis, atrophy or volume loss in the hippocampus or overt mesial temporal sclerosis (MTS) or similar relevant anatomical pathology; the detection in the patient's blood, serum or tissues of a molecular species such as a protein or other biochemical biomarker, e.g., elevated levels of ciliary neurotrophic factor (CNTF) or elevated serum levels of a neuronal degradation product; or other evidence from surrogate markers or biomarkers that the patient is in need of treatment with a neuroprotective drug.

A determination that a subject has, or may be at risk for developing, neuronal damage would also include, for example, a medical evaluation that includes a thorough history, a physical examination, and a series of relevant bloods tests. It can also include an electroencephalogram (EEG), CT, MRI or PET scan. A determination of an increased risk of developing neuronal damage or injury may also be made by means of genetic testing, including gene expression profiling or proteomic techniques. For psychiatric disorders that may be stabilized or improved by a neuroprotective drug, e.g., bipolar disorder, schizoaffective disorder, schizophrenia, impulse control disorders, etc. the above tests may also include a present state exam and a detailed history of the course of the patients symptoms such as mood disorder symptoms and psychotic symptoms over time and in relation to other treatments the patient may have received over time, e.g., a life chart. These and other specialized and routine methods allow the clinician to select patients in need of therapy using the methods and formulations of this invention. In some embodiments of the present invention ozone agent suitable for use in the practice of this invention will be administered either singly or concomitantly with at least one or more other compounds or therapeutic agents, e.g., with other neuroprotective drugs or antiepileptic drugs, anticonvulsant drugs. In these embodiments, the present invention provides methods to treat or prevent neuronal injury in a patient. The method includes the step of; administering to a patient in need of treatment, an effective amount of one of the ozonedisclosed herein in combination with an effective amount of one or more other compounds or therapeutic agents that have the ability to provide neuroprotection or to treat or prevent seizures or epileptogenesis or the ability to augment the neuroprotective effects of the compounds of the invention.

As used herein the term “combination administration” of a compound, therapeutic agent or known drug with the therapeutic ozone agent of the present invention means administration of the drug and the one or more compounds at such time that both the known drug and the therapeutic ozone agent will have a therapeutic effect. In some cases this therapeutic effect will be synergistic. Such concomitant administration can involve concurrent (i.e. at the same time), prior, or subsequent administration of the drug with respect to the administration of the therapeutic ozone agent of the present invention. A person of ordinary skill in the art would have no difficulty determining the appropriate timing, sequence and dosages of administration for particular drugs and ozoneof the present invention.

The said one or more other compounds or therapeutic agents may be selected from compounds that have one or more of the following properties: antioxidant activity; NMDA receptor antagonist activity, augmentation of endogenous GABA inhibition; NO synthase inhibitor activity; iron binding ability, e.g., an iron chelator; calcium binding ability, e.g., a Ca (II) chelator; zinc binding ability, e.g., a Zn (II) chelator; the ability to effectively block sodium or calcium ion channels, or to open potassium or chloride ion channels in the CNS of a patient.

Heart and Vascular Disease

Heart disease is a general term used to describe many different heart conditions. For example, coronary artery disease, which is the most common heart disease, is characterized by constriction or narrowing of the arteries supplying the heart with oxygen-rich blood, and can lead to myocardial infarction, which is the death of a portion of the heart muscle. Heart failure is a condition resulting from the inability of the heart to pump an adequate amount of blood through the body. Heart failure is not a sudden, abrupt stop of heart activity but, rather, typically develops slowly over many years, as the heart gradually loses its ability to pump blood efficiently. Risk factors for heart failure include coronary artery disease, hypertension, valvular heart disease, cardiomyopathy, disease of the heart muscle, obesity, diabetes, and/or a family history of heart failure.

Examples of cardiovascular diseases and disorders are: aneurysm, stable angina, unstable angina, angina pectoris, angioneurotic edema, aortic valve stenosis, aortic aneurysm, arrhythmia, arrhythmogenic right ventricular dysplasia, arteriosclerosis, arteriovenous malformations, atrial fibrillation, Behcet syndrome, bradycardia, cardiac tamponade, cardiomegaly, congestive cardiomyopathy, hypertrophic cardiomyopathy, restrictive cardiomyopathy, carotid stenosis, cerebral hemorrhage, Churg-Strauss syndrome, diabetes, Ebstein's Anomaly, Eisenmenger complex, cholesterol embolism, bacterial endocarditis, fibromuscular dysplasia, congenital heart defects, heart diseases, congestive heart failure, heart valve diseases, heart attack, epidural hematoma, hematoma, subdural, Hippel-Lindau disease, hyperemia, hypertension, pulmonary hypertension, cardiac hypertrophy, left ventricular hypertrophy, right ventricular hypertrophy, hypoplastic left heart syndrome, hypotension, intermittent claudication, ischemic heart disease, Klippel-Trenaunay-Weber syndrome, lateral medullary syndrome, long QT syndrome mitral valve prolapse, moyamoya disease, mucocutaneous lymph node syndrome, myocardial infarction, myocardial, ischemia, myocarditis, pericarditis, peripheral vascular diseases, phlebitis, polyarteritis nodosa, pulmonary atresia, Raynaud disease, Sneddon syndrome, superior vena cava syndrome, syndrome X, tachycardia, Takayasu's arteritis, hereditary hemorrhagic telangiectasia, telangiectasis, temporal arteritis, tetralogy of Fallot, thromboangiitis obliterans, thrombosis, thromboembolism, tricuspid atresia, varicose veins, vascular diseases, vasculitis, vasospasm, ventricular fibrillation, Williams syndrome, peripheral vascular disease, varicose veins and leg ulcers, deep vein thrombosis, Wolff-Parkinson-White syndrome.

Vascular diseases are often the result of decreased perfusion in the vascular system or physical or biochemical injury to the blood vessel.

Peripheral vascular disease (PVD) is defined as a disease of blood vessels often encountered as narrowing of the vessels of the limbs. There are two main types of these disorders, functional disease which doesn't involve defects in the blood vessels but rather arises from stimuli such as cold, stress, or smoking, and organic disease which arises from structural defects in the vasculature such as atherosclerotic lesions, local inflammation, or traumatic injury. This can lead to occlusion of the vessel, aberrant blood flow, and ultimately to tissue ischemia.

One of the more clinically significant forms of PVD is peripheral artery disease (PAD). PAD is often treated by angioplasty and implantation of a stent or by artery bypass surgery. Clinical presentation depends on the location of the occluded vessel. For example, narrowing of the artery that supplies blood to the intestine can result in severe postprandial pain in the lower abdomen resulting from the inability of the occluded vessel to meet the increased oxygen demand arising from digestive and absorptive processes. In severe forms the ischemia can lead to intestinal necrosis. Similarly, PAD in the leg can lead to intermittent pain, usually in the calf, that comes and goes with activity. This disorder is known as intermittent claudication (IC) and can progress to persistent pain while resting, ischemic ulceration, and even amputation.

Peripheral vascular disease is also manifested in atherosclerotic stenosis of the renal artery, which can lead to renal ischemia and kidney dysfunction.

One disease in which vascular diseases and their complications are very common is diabetes mellitus. Diabetes mellitus causes a variety of physiological and anatomical irregularities, the most prominent of which is the inability of the body to utilize glucose normally, which results in hyperglycemia. Chronic diabetes can lead to complications of the vascular system which include atherosclerosis, abnormalities involving large and medium size blood vessels (macroangiopathy) and abnormalities involving small blood vessels (microangiopathy) such as arterioles and capillaries.

Patients with diabetes mellitus are at increased risk of developing one or more foot ulcers as a result of established long-term complications of the disease, which include impaired nerve function (neuropathy) and/or ischemia. Local tissue ischemia is a key contributing factor to diabetic foot ulceration.

In addition to large vessel disease, patients with diabetes suffer further threat to their skin perfusion in at least two additional ways. First, by involvement of the non-conduit arteries, which are detrimentally affected by the process of atherosclerosis, and secondly, and perhaps more importantly, by impairment of the microcirculatory control mechanisms (small vessel disease). Normally, when a body part suffers some form of trauma, the body part will, as part of the body's healing mechanism, experience an increased blood flow. When small vessel disease and ischemia are both present, as in the case of many diabetics, this natural increased blood flow response is significantly reduced. This fact, together with the tendency of diabetics to form blood clots (thrombosis) in the microcirculatory system during low levels of blood flow, is believed to be an important factor in ulcer pathogenesis.

Neuropathy is a general term which describes a disease process which leads to the dysfunction of the nervous system, and is one of the major complications of diabetes mellitus, with no well-established therapies for either its symptomatic treatment or for prevention of progressive decline in nerve function.

The thickening and leakage of capillaries caused by diabetes primarily affect the eyes (retinopathy) and kidneys (nephropathy). The thickening and leakage of capillaries caused by diabetes are also associated with skin disorders and disorders of the nervous system (neuropathy).

The eye diseases associated with diabetes are nonproliferative diabetic retinopathy, proliferative diabetic retinopathy, diabetic maculopathy, glaucoma, cataracts and the like.

Other diseases, although not known to be related to diabetes are similar in their physiological effects on the peripheral vascular system. Such diseases include Raynaud syndrome, CREST syndrome, autoimmune diseases such as erythematosis, rheumatoid disease, and the like.

As used herein, the term “peripheral vascular diseases” comprises any peripheral vascular disease including peripheral and autonomic neuropathies. Examples of “peripheral vascular disease” include peripheral arterial disease, such as chronic arterial occlusion including arteriosclerosis, arteriosclerosis obliterans and thromboangiitis obliterans (Buerger's disease), macroangiopathy, microangiopathy, diabetes mellitus, thrombophlebitis, phlebemphraxis, Raynaud's disease, Raynaud's syndrome, CREST syndrome, health hazard due to vibration, Sudeck's syndrome, intermittent claudication, cold sense in extremities, abnormal sensation in extremities, sensitivity to the cold, Meniere's disease, Meniere's syndrome, numbness, lack of sensation, anesthesia, resting pain, causalgia (burning pain), disturbance of peripheral circulation function, disturbance of nerve function, disturbance of motor function, motor paralysis, diabetic peripheral circulation disorder, lumbar spinal canal stenosis, diabetic neuropathy, shock, autoimmune disease such as erythematosis, rheumatoid disease and rheumatoid arthritis, autonomic neuropathy, diabetic autonomic neuropathy, autonomic imbalance, orthostatic hypotension, erectile dysfunction, female sexual dysfunction, retrograde ejaculation, cystopathy, neurogenic bladder, defective vaginal lubrication, exercise intolerance, cardiac denervation, heat intolerance, gustatory sweating, diabetic complication, hyperglycemia, hypoglycemia unawareness, hypoglycemia unresponsiveness; glaucoma, neovascular glaucoma, cataract, retinopathy, diabetic retinopathy, diabetic maculopathy, occlusion of retinal artery, obstruction of central artery of retina, occlusion of retinal vein, macular edema, aged macular degeneration, aged disciform macular degeneration, cystoid macular edema, palpebral edema, retinal edema, chorioretinopathy, neovascular maculopathy, uveitis, iritis, retinal vasculitis, endophthalmitis, panophthalmitis, metastatic ophthalmia, choroiditis, retinal pigment epithelitis, conjunctivitis, cyclitis, scleritis, episcleritis, optic neuritis, retrobulbar optic neuritis, keratitis, blepharitis, exudative retinal detachment, corneal ulcer, conjunctival ulcer, chronic nummular keratitis, Thygeson keratitis, progressive Mooren's ulcer, damage of skin, skin ulcer including foot ulcer, diabetic ulcer, burn ulcer, lower leg ulcer, postoperative ulcer, traumatic ulcer, ulcer after herpes zoster, radiation ulcer, drug induced ulcer, frostbite (cold injury), chilblain, gangrene and sudden gangrene, angina pectoris/variant angiitis, coronary arteriosclerosis (chronic ischemic heart disease, asymptomatic ischemic heart disease, arteriosclerotic cardiovascular disease), myocardial infarction, heart failure, congestive heart failure and painless ischemic heart disease, pulmonary edema, hypertension, pulmonary hypertension; portal hypertension, diabetic nephropathy, decubitus, renal failure.

The invention will be illustrated in more detail with reference to the following Examples, but it should be understood that the present invention is not deemed to be limited thereto.

### EXAMPLES

### Example 1

Sickle cell anemia, a genetic disease which involves the sickle shape of erythrocytes, when blood oxygen tension is low, is represented by a modified hemoglobin, HbS, due to the substitution of glutamic acid by valine in the amino acid chain. The crystallization or intracellular polymerization of the molecules of HbS occurs when those cells are deprived of oxygen up to a partial pressure of oxygen (pO2) below the threshold level at which the cells sickle. In these conditions, the erythrocytes lose their normal elasticity and shape, also losing their capacity to take and deliver oxygen and increasing the viscosity of the blood. This leads to a reduction in the availability of oxygen to the cells, producing painful crisis, infarction, abdominal and/or muscular pain, ulcers, etc. This process is reversible in the early stages, for when the HbS molecule is reoxygenated the cell distortion disappears and resumes its normal shape. The longer the period of time necessary for the reoxy genation of the molecules of HbS, the greater number of red cells die. Therefore, the increase in the partial pressure of oxygen and the promptness with which it is normalized are determining factors for the symptoms to diminish and disappear. (Aluoch J R. The treatment of sickle cell disease. A historical and chronological literature. Review of the therapies applied since 1910. Supplement to Tropical and Geographical Medicine. Vol. 36, No. 4, 1984) Attempts have been made to establish effective treatment for this disease, but their value and absence of side effects has not been confirmed in medical practice. (Reynolds J D H. Painful sickle cell crisis: successful treatment with hyperbaric oxygen therapy. JAMA; 1971; 216: 1977-8; Sergeant G R. Sickle cell disease. Oxford University Press. p. 48, 1985; Reinhard E H., Moore C V., Dubach R, Wade L J. Depressant effect of high concentrations of inspired oxygen on erythrocytogenesis. Observations on patients with sickle cell anemia with a description of the observed toxic manifestations of oxygen. Jour. Clin. Inv., vol. 23 p. 682-98, 1944.).

Numerous reports have been published on the safety and clinical results obtained by the application of medical ozone/oxygen in diseases related to insufficient oxygen supply to tissues and various organs, and/or the disruption of its utilization in the cells. Ozone virucidal effect has been reported at dose levels at which no undesirable side effects take place, offering promise as a means to inactivate human retroviruses in human body fluids and blood products preparation.

Among the medical properties of ozone documented are the ability to increase the rate and capacity of oxygen absorption in erythrocytes and the activation of glycolisis in the cells via the pentose pathway. This enhances the production of 2,3 DPG, which is known to act as a coadjuvant of oxygen release from oxyhemoglobin at tissue level. (Wells K H., Latino J., Gavalchin J. Inactivation of human immunodeficiency virus type I by ozone in vitro. Blood, vol. 78:7, p. 1982, 1991; Balkanyi A. The interaction between ozone therapy and oxygen radicals in their importance in practice. Proceedings of the Ninth Ozone World Congress. New York. Jun. 3-9, 1989.) Both effects lead to significant improvement in oxygen supply to the body, demonstrated in vivo by the measurement of pO2 increase in arterial blood as well as the reduction in venous. (Baltin H. Oxygen partial pressure measurements in the arterial and venous blood before, during and after ozone treatment. OzoNews 2 (2):41, 1983). In addition, the rheological properties of the blood improve, especially in regard to erythrocytes aggregation (preventing rouleaux formation and clumping) and membrane permeability and flexibility, because of the effect of ozone/oxygen on it. As a consequence of these effects, reduction of blood viscosity and enhancement of blood flow are achieved. (Rokitansky O. Clinical considerations and Biochemistry of ozone therapy. Hospitalis, 52,643, 1982; Gomez M., Menendez S. About ozone metabolic effects in biological systems. (Spa.). Conference presented in the International Congress Geriatrics/92, Palace of Conventions, Ciudad Habana, Mayo 1992; Mattasi, R. Ozonoterapia. Organizzazione Editoriale Medico Farmaceutica, Milano 1985, p 64.).

Numerous preclinical experiments have been performed in vitro and in vivo to test possible ozone/oxygen toxicity related to the therapeutic methods and ways of administration according to which medical ozone/oxygen is currently applied. Controlled in vitro testing on the degree of hemolysis and “Heinz Body Formation” induced by the administration of ozone to blood at adequate dosage was performed, (Hernandez F., Menendez S., Gomez M. Blood ozone treatment and the hemolysis level. X Ozone World Congress. Monaco, 1991) not finding any significant effect, neither on the hemolysis level nor in the resistance of erythrocytes to further oxidative stress. These results are consistent with the fact that ozone stimulates several enzymatic redox systems responsible for cells protection against oxidation.

Doses up to more than ten times the maximum therapeutic levels were tested for toxicity. These studies (Noa M., Fernadez M., Herrera M., Menendez S., Capote A. y Aguilar C. Morphological observations in rats treated with ozone by intramuscular applications. Proceedings of the 10th Ozone World Congress. Monaco. Vol. 3, p. 35, 1991; Fernandez, S. I., Quinzan C., Menendez S. Gomez M. y Acosta P. Study of the effect of intratesticular injection of ozone on spermatogenesis. (Spa.) Proceedings of the I National Conference on Ozone Applications (La Habana, 9-10 Dec. 1988). Rev. CENIC (Ciencias Biologicas). Vol. 20, p. 16, 1-2-3. enero-diciembre 1989; Fernandez, S. I., Quinzan C., Menendez S. Gomez M. Study in Lab Animals about possible teratogenic and mutagenic effects by intra-peritoneal and intra-muscular administration. (Spa.) Proceedings I National Conference on Ozone applications (La Habana, 9-10 Dec. 1988). Rev. CENIC (Ciencias Biologicas). Vol. 20, p. 45, 1-2-3. enero-diciembre 1989; Rodriguez M. D. Menendez S. Gomez M., Garcia H. Teratogenic Study of ozone administered by rectal insufflation. (Spa.) Proceedings I National Conference on Ozone applications (La Habana, 9-10 Dec. 1988). Rev. CENIC (Ciencias Biologicas). Vol. 20, p. 45, 1-2-3. enero-diciembre 1989; Friman M., Walker D. Menendez S., Gomez M. y En, L. Effect of ozone on circulating endothelium cells in rats. (Spa.) Proceedings I National Conference on Ozone Applications (La Habana, 9-10 Dec. 1988). Rev. CENIC (Ciencias Biologicas). Vol. 20, p. 45, 1-2-3. enero-diciembre 1989. Rev. CENIC (Ciencias Biologicas). Vol. 20, p. 25; enero-diciembre 1989; Rokitansky O and Rokitansky, A. Electron microscopic studies on capillary endothelium cells and on the peritoneum after application of ozone-oxygen administered in animals. Proceedings of the 8th Ozone World Congress. Zurich, Switzerland, 1987; De las Cagigas A., Perez., Menendez S. y Gomez M. Mutagenic study of ozone administered by autohemotherapy. (Spa.) Proceedings I National Conference on Ozone Applications (La Habana, 9-10 Dec. 1988). Rev. CENIC (Ciencias Biologicas). Vol. 20, p. 41, 1-2-3. enero-diciembre 1989; Prieto E., Mendez D., Menendez S. Gomez M. Montejo L. y Johnson J. Evaluation of ozone genotoxicity through citogenetic methods. (Spa.) First Iberolatinamerican Congress on Ozone Applications. La Habana, 31 Oct.-3 Nov. 1990) comprised cytotoxicity, organs function, hematological parameters, histological studies by electron microscopy, teratogenicity and cytogenetic testing. All the results demonstrated the non-toxic nature of medical ozone/oxygen within the range of therapeutic dose levels when administered intravascularly, intramuscularly and intrarectally.

Based on the major role blood deoxygenation and hypoxia play in the onset and persistence of painful sickle cell crisis, and considering the established therapeutic properties of medical ozone/oxygen, and the absence of negative side effects, an evaluation of the possible effectiveness of this treatment for the prevention and/or the timely resolution of the crisis was made, by means of controlled in vitro and clinical trials. It was encouraged by our extensive successful practice in numerous Havana City Hospitals, fundamentally that regarding the treatment of patients suffering circulatory insufficiencies, diabetes and many other diseases related to insufficient supply of oxygen to tissue. (Proceedings of the First Iberolatinamerican Congress on Ozone Applications. La Habana, 31 Oct.-3 Nov. 1990)

### Materials and Methods

Ozone/oxygen was obtained from a pure (medical grade) oxygen by means of a medical ozone generator (Ozomed, a device manufactured in N.C.S.R., Havana, Cuba), at a concentration of 50 mg ozone/L oxygen (ca. 3.7%).

The ability of ozone/oxygen to in vitro raise pO2 of normal bank blood was compared to that of pure medical oxygen. To each one often paired (5+5) blood samples (5 mL each) contained in closed 10 ml vials, 2.5 mL of pure oxygen or ozone/oxygen mixture respectively were carefully bubbled through. Gasometric analyses were performed in test samples 5 minutes after gas administration and also in control untreated samples.

Preliminary clinical trials were performed comparing two modes of administration of medical ozone/oxygen: autohemotherapy (5 mg) and rectal insufflation (10 mg). Similar responses were obtained in both groups. Taking into account these results and considering the frequent difficulties encountered with the veins of these sickle cell patients, the intra rectal mode was chosen. Ozone was administered daily (5 days per week) for 3 weeks. For the controlled clinical trial, 55 adult sickle cell anemia patients were studied, each suffering from painful crisis in different degrees of intensity, who were admitted to the emergency service of the Institute of Hematology and Immunology, Havana, Cuba. Informed consent was gotten from patients before entering the study. After resolution of the crises, a 6-month follow-up was performed in every patient. Two groups were established with patients selected at random:

Group 1: Control group, comprised of 25 patients who received conventional treatment with analgesics, vasodilators, and i.v. hydration (saline solution).

Group 2: Ozone/oxygen treated group, comprising 30 patients who received the same treatment plus ozone therapy.

TABLE 1
Distribution by Sex and Age

| | Sex | | | Mean Age (range) | |
|---|---|---|---|---|---|
| | Male | Female | Total | Male | Female |
| Group 1 | 11 | 14 | 25 | 26 (22-34) | 25 (21-30) |
| Group 2 | 12 | 18 | 30 | 28 (30-36) | 26 (21-32) |

The distribution of patients by sex and age in both groups is presented in Table 1.

There were not significant differences in age and sex among groups. Also both had similar mean values of hemoglobin, reticulocytes, fetal hemoglobin, hemoglobin S and oxygen partial pressure (pO2). No patient received a blood transfusion in the period of 6 months prior to this study. Severity of crisis were classified according to:

Mild crisis: Lasting not more than 12 hours; treated with analgesics, not needing i.v. saline solution for relief. Relatively low intensity pain, located in one or two body regions.

Moderate crisis: Within 12 and 24 hours duration. In addition to analgesics, also requiring continuous i.v. saline solution for relief. Moderate pain, localized in two or more regions in the body.

Severe crisis: More than 24 hours duration. Requiring analgesics, continuous i.v. saline solution and other therapeutic procedures (including whole blood transfusions). Intensive pain localized in 3 or more regions in the body.

### Results and Discussion

The in vitro experiments with stored venous blood demonstrated considerably higher capacity for oxygen absorption in the samples treated with ozone/oxygen. These results are reported in Table 2:

TABLE 2
Values of PO2 and Hemoglobin Saturation of Blood and the Same
Blood After Oxygen or Ozone/Oxygen Treatment In Vitro

| | Control | O2 Treated | O3/O2 Treated |
|---|---|---|---|
| | n = 5 | n = 5 | n = 5 |
| PO2 | 41 ± 1 | 73 ± 2 | 100 ± 2 |
| PO2 rise (%) | — | 78 | 143 |
| (above control) | 60 ± 1 | 86 ± 2 | 93 ± 3 |
| HB saturation | | | |

Table 2 shows pO2 and hemoglobin saturation values in control untreated blood, and those achieved in samples of the same blood after respective oxygen or ozone/oxygen administration. The relative rise (%) in pO2 obtained with ozone/oxygen nearly doubled that obtained with only pure oxygen. There were no significant differences in pH values among samples.

In the controlled clinical study comprising the aforementioned 55 patients, several hematological parameters were evaluated before and after treatment. These results are shown in Table 3:

TABLE 3
Parameters Measured in Arterial Blood of Patients of Both Groups Before and After Treatment

| | Group 1 (25 patients) | | | | | | Group 2 (30 patients) | | | | | |
|---|---|---|---|---|---|---|---|---|---|---|---|---|
| | Initial | | | Final | | | Initial | | | Final | | |
| | x̅ | | SD | x̅ | | SD | x̅ | | SD | x̅ | | SD |
| pO2 (mm Hg) | 71.2 | ± | 3.1 | 72.5 | ± | 2.5 | 71.5 | ± | k | *84 | ± | 4 |
| Hemoglobin (g/dL) | 7.7 | ± | 0.5 | 7.7 | ± | 0.4 | 8.0 | ± | 0.5 | 8.4 | ± | 0.4 |
| Reticulocytes (%) | 10 | ± | 4 | 9.4 | ± | 5.0 | 9.4 | ± | 4.0 | 11 | ± | 3 |
| Hemoglobin S (%) | 95 | ± | 3 | 95 | ± | 2 | 97 | ± | 3 | 96 | ± | 2 |

*p < 0.05

Mean arterial blood pO2 value in control group patients remained virtually unchanged whereas a significant increase (17%) was achieved for this parameter in group 2 (ozone/oxygen treated) patients after 15 treatments. The remaining blood parameters did not show any significant change in any group. It should be noted that the rise in pO2 was obtained with no reduction in hemopoiesis, (hemoglobin values remained stable), the latter reported to occur when hyperbaric oxygen is administered. Thus, ozone/oxygen therapy improved oxygen transport to tissue without adverse effects on the blood.

At the moment of inclusion in the study, the crises of patients included in the control and treated groups presented the characteristics shown in Table 4:

TABLE 4
Distribution of Painful Crises Severity in Patients of
Both Groups at the Inclusion of the Study

| | Mild % | Moderate % | Severe % |
|---|---|---|---|
| Group 1—25 | 13-52% | 6-24.0% | 6-24.0% |
| Control | | | |
| Group 2—30 | 13-43% | 10-33.3% | 7-23.3% |
| Ozone | | | |

It was clinically observed, that by virtue of the better reoxygenation of blood in ozone-treated patients, demonstrated objectively by the rise in arterial blood pO2 (shown in Table 3) the promptness with which all crises (whether mild, moderate or severe) were resolved was significantly accelerated in the ozone/oxygen treated group as compared to the control group. These values are shown in Table 5:

TABLE 5
Average Time for Resolution of Painful Crises in Patients (hours)

| | mild | moderate | severe |
|---|---|---|---|
| Group 1 (25) | 8 | 16 | 52 |
| (control) | | | |
| Group 2 (30) | 4 | 9 | 25 |
| (ozone) | | | |

TABLE 6
Average Number of Painful Crises per Patient in the Six
Month Follow-up for those Receiving Conventional
Treatment (Control) or Ozone/Oxygen Therapy

| | mild % | moderate % | severe % | Total |
|---|---|---|---|---|
| Group 1 (25) | 2.5 (49.0) | 1.2 (23.5) | 1.4 (27.5) | 5.1 |
| (control) | | | | |
| Group 2 (30) | 1.2 (48.0) | 1.0 (40.0) | 0.3 (12.0) | 2.5 |
| (ozone) | | | | |

After resolution of the initial crisis, ozone/oxygen treatments were continued prophylactically. They were administered one every 14 days over a period of 6 months to patients in the treated group. The number of subsequent crises per patient in this period were significantly smaller in the ozone/oxygen treated group, compared to the control group (receiving conventional therapy).

The proportion of severe crises also diminished very sharply in the treated group, compared to the initial proportion.

There were no adverse reactions observed during or after the administration of ozone in any of the patients treated. Additionally, it is of interest to mention that some of the patients in the treated group who suffered from associated clinical manifestations including: acute severe chest syndrome, priapism and duodenal ulcer, showed remarkable favorable resolution of such conditions.

### CONCLUSIONS

Medical ozone significantly raises blood pO2 in vitro.

Ozone/oxygen therapy produces significant rise in arterial blood pO2 of patients, which is an objective effect, without significant alterations in other blood parameters. It does not inhibit hemopoiesis, as is the case with hyperbaric oxygen treatment.

The average time required for resolution of painful sickle cell crises (mild, moderate and severe) in ozone treated patients was about half of that required to resolve painful crisis in control patients.

PO2 values in the ozone/oxygen treated group during the follow-up remained high enough to significantly reduce the incidence of crisis as compared to the control group, regardless of whether their crises were classified as mild, moderate or severe.

Frequency and severity of painful crises in sickle cell anemia patients receiving ozone/oxygen therapy during the six month follow-up was significantly lower in comparison with control group patients.

No adverse reactions were observed objectively or subjectively in the patients who received ozone/oxygen therapy.

What is claimed is:

1. A method for treating or preventing carbon monoxide poisoning in a patient wherein said method comprises:
selecting a patient in need of treating or preventing carbon monoxide poisoning;
administering to the patient in need of treating or preventing carbon monoxide poisoning at least one therapeutic ozone agent in a therapeutically effective amount.

2. The method of claim 1, wherein the carbon monoxide poisoning is selected from the group consisting of acute, chronic, and combinations thereof.

3. The method of claim 1, wherein the a therapeutic ozone agent is administered by a method selected from the group consisting of rectal insufflation, extracorporeal treatment and re-administration of the patient's blood, intramuscular administration, cardiopulmonary bypass, and combinations thereof.

4. The method of claim 1, wherein the at least one therapeutic ozone agent is administered in a form selected from the group consisting of a gas, a liquid formulation, a combination with O2, and combinations thereof.

5. A method for treating carbon monoxide poisoning in a patient wherein said method comprises:
selecting a patient in need of treatment of carbon monoxide poisoning;
administering to the patient in need of treatment of carbon monoxide poisoning a therapeutic ozone agent in a therapeutically effective amount, wherein the therapeutically effective amount of the therapeutic ozone agent is administered in a concentration to convert carbon monoxide dissolved in the patient's plasma to carbon dioxide, and also to convert carbon monoxide bonded to the patient's hemoglobin receptors to carbon dioxide, wherein the ozone is administered over a period of time sufficient to diminish CO levels in the plasma, further wherein the ozone administered causes a rise in PO₂ from between the range of 2% to 30% thereby treating said carbon monoxide poisoning in said patient.

6. The method of claim 5 wherein the therapeutic ozone agent is ozone alone.

7. The method of claim 5, wherein the carbon monoxide poisoning is selected from the group consisting of acute, chronic, and combinations thereof.

8. The method of claim 5, wherein the a therapeutic ozone agent is administered by a method selected from the group consisting of rectal insufflation, extracorporeal treatment and re-administration of the patient's blood, intramuscular administration, cardiopulmonary bypass, and combinations thereof.

9. The method of claim 5, wherein the at least one therapeutic ozone agent is administered in a form selected from the group consisting of a gas, a liquid formulation, a combination with O₂, and combinations thereof.

10. The method of claim 5 wherein the dose therapeutically effective amount of therapeutic ozone administered is varied according to individual's sensitivity and the type of disease state being treated.
